(19) United States
(12) Patent Application Publication
(10) Pub. No.: US 2014/0066411 A1
(43) Pub. Date: Mar. 6, 2014

(54) PHARMACEUTICAL COMPOSITIONS FOR EXTENDED RELEASE OF AZO-BONDED 5-AMINOSALICYLIC ACID COMPOSITIONS

(71) Applicant: Depomed, Inc., Newark, CA (US)

(72) Inventors: Verne Earle Cowles, Dublin, CA (US); Ryan Douglas Fell, San Carlos, CA (US)

(73) Assignee: Depomed, Inc., Newark, CA (US)

(21) Appl. No.: 14/012,484

(22) Filed: Aug. 28, 2013

Related U.S. Application Data
(60) Provisional application No. 61/694,164, filed on Aug. 28, 2012.

(51) Int. Cl. A61K 47/34

(52) U.S. Cl. CPC A61K 47/34
USPC 514/150

(57) ABSTRACT

Gastric retentive dosage forms for sustained release of an azo-bonded prodrug of 5-aminosalicylic acid (5-ASA) are described which may allow once- or twice-daily dosing for both acute and long-term treatment of inflammatory bowel disorders. Methods of treatment using the dosage forms and methods of making the dosage forms are also described.

### CROSS-REFERENCE TO RELATED APPLICATIONS

This application claims the benefit of U.S. Provisional Application No. 61/694,164, filed on Aug. 28, 2012, the contents of which are incorporated by reference herein it its entirety.

### TECHNICAL FIELD

The present subject matter relates generally to dosage forms for extended release of a prodrug of 5-aminosalicylic acid (5-ASA) containing an azo bond, such as olsalazine, sulfasalazine or balsalazide, into the stomach of a patient in the fed mode and to methods of treatment using the dosage forms.

### BACKGROUND

Ulcerative colitis (UC) is an idiopathic chronic inflammatory disease of the colon which requires both acute and long-term medical therapy to induce and maintain remission (Sanborn and Hanauer, 2003, Aliment Pharmacol Ther, 17:29-42). Whereas Crohn's disease affects both the large and small intestine, UC is limited to the colon. Moreover, effective treatment requires at least a topical exposure of the inflamed tissue to the active agent. Currently, the first-line therapy for UC is 5-aminosalicylic acid (5-ASA or mesalamine), an anti-inflammatory agent. While the ability of 5-ASA to reduce inflammation is well established, its success is limited due to the inability to consistently release the 5-ASA to the inflamed tissue of the colon without inducing significant adverse side effects.

To overcome this problem, prodrugs have been developed which resist stomach acidity, and are not absorbed in the small intestine, but release free 5-ASA in the colon. Each of the prodrugs consists of a molecule of 5-ASA joined by an azo bond to a moiety which prevents small-bowel absorption. However, once the prodrug is exposed to the microflora of the colon, it is converted by the bacterial azo-reductase activity to 5-ASA, thus allowing delivery of the active agent primarily to the colon. One such prodrug is olsalazine, shown below.

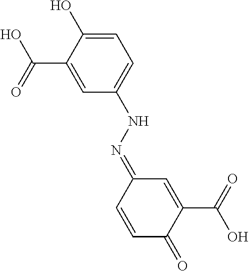 [SMILES: O=C(O)C1=C/C(=N\Nc2ccc(O)c(C(=O)O)c2)C=CC1=O]

Olsalazine is currently marketed as Dipentum® (Pharmacia Corp.). It is an immediate release tablet administered twice a day, usually at a dose of about 500 mg per administration. Similar marketed prodrugs include sulfasalazine (e.g., Azulfidine®) and balsalazide (e.g., Giazo® and Colazal®).

Once the prodrug is converted to 5-ASA in the colon, the 5-ASA is only minimally absorbed. The majority is excreted unchanged in the feces. 5-ASA acts through direct contact with the colonic mucosa to suppress various pro-inflammatory pathways including both cyclooxygenase and lipoxygenase derived products such as prostaglandins and leukotrienes from arachidonic acid and from suppression of superoxide dismutase.

Although the 5-ASA prodrugs have proven both safe and effective for short-term and long-term use, there remains a need to both optimize exposure of the inflamed colonic tissue to the active agent as well as reduce the adverse side effects of these prodrugs.

Concentrations of 5-ASA released from the prodrug may be reduced to ineffective levels due to a lack of conversion of the prodrug to 5-ASA. This may be caused by a lack of bacteria which provide the needed azo-reductase activity or the presence of frequent diarrhea in UC patients, resulting in rapid transit of the prodrug through the colon. Furthermore, olsalazine has been shown to cause a concentration dependent secretory response in the ileum that results in decreased water uptake in the small intestine followed by increased incidence of diarrhea.

Thus it would be useful to manufacture a dosage form which is able to provide prolonged and steady levels of 5-ASA to the colon at concentrations effective for reducing and/or preventing inflammation without eliciting the adverse side effects. Particularly advantageous would be a gastric retentive oral dosage form which provide an extended release of the prodrug into the stomach such that a continuous flow of the prodrug is provided to the colon, with the generation of active 5-ASA upon entrance of the prodrug into the colon.

Gastric retained forms that can form the basis for the sustained release of a prodrug have been previously described, for example, in Gusler et al. (U.S. Pat. No. 6,723,340), Berner et al. (U.S. Pat. No. 6,488,962), Shell et al., (U.S. Pat. No. 6,340,475) and Shell et al. (U.S. Pat. No. 6,635,280). These formulations make use of one or more hydrophilic polymers which swell upon intake of water from gastric fluid. Thus, when administered in the fed mode, when the size of pyloric sphincter is reduced, the dosage form will swell to a size to be retained in the stomach for a minimum of four hours or more. These formulations may be designed to produce desired release and delivery profiles for both highly soluble and poorly soluble drugs.

As presently disclosed, gastric retentive dosage forms are formulated specifically to provide extended exposure of colonic tissue to a therapeutically effective concentration of 5-ASA as a means to reduce and prevent inflammation of colonic tissue while minimizing adverse side effects commonly associated with the administration of azo-bonded prodrugs.

### BRIEF SUMMARY

The present disclosure provides, among other aspects, gastric retentive dosage forms for oral administration to a subject, such as a human patient, for the treatment of inflammatory bowel disorders including, but not limited to, ulcerative colitis (UC). The dosage form in some embodiments is a gastric retentive dosage form that contains a dose of an azo-bonded prodrug of 5-aminosalicylic acid (5-ASA) in an extended release (“ER”) formulation.

In a first aspect, an oral dosage form comprising a dose of an azo-bonded prodrug of 5-ASA dispersed in a polymer matrix comprising at least one hydrophilic polymer is provided. Upon administration, the polymer matrix swells upon imbibition of fluid to a size sufficient such that the dosage form is retained in the stomach of a subject and the dose of azo-bonded prodrug of 5-ASA is released over an extended period of time.

In one embodiment, the azo-bonded prodrug of 5-ASA is olsalazine, sulfasalazine or balsalazide.

In one embodiment, the oral dosage form is a tablet. In another embodiment, the total tablet weight is about 1000 mg (milligrams). In still another embodiment, the total tablet weight is about 1200 mg. In yet another embodiment, the total tablet weight is about 500 mg to about 2000 mg, 750 mg to 1500 mg, 800 mg to 1300 mg, 900 mg to 1250 mg, or about 800 to 1200 mg.

In one embodiment, the tablet comprises a total of about 200 mg to about 1000 mg, or about 500 mg to about 750 mg azo-bonded prodrug of 5-ASA. In another embodiment, the tablet comprises about 300 mg to 850 mg, 350 mg to 800 mg, 400 mg to 700 mg, 250 mg to 500 mg, 500 mg to 1000 mg, 700 mg to 1000 mg, 400 mg to 800 mg, 500 mg to 900 mg, 450 mg, 550 mg, 600 mg, 650 mg, 700 mg, 750 mg, 800 mg, or 850 mg of the prodrug. In yet another embodiment, the tablet comprises about 50 wt % (weight percent) or about 75 wt % of the prodrug. In still another embodiment, the tablet comprises about 30 wt %, 35 wt %, 40 wt %, 45 wt %, 55 wt %, 60 wt %, 65 wt %, 70 wt %, 75 wt % or about 80 wt % of the prodrug.

In one embodiment, the tablet comprises about 20 mg, 25 mg, 30 mg, 35 mg, 40 mg, 45 mg, or 50 mg of one or more binders. In another embodiment, the tablet comprises about 1 wt %, 2 wt %, 2.5 wt %, 3 wt %, 3.5 wt %, 4 wt %, 4.5 wt %, 5 wt %, or 6 wt % binder. In yet another embodiment, the tablet comprises about 1 wt % to about 6 wt % or about 2 wt % to about 5 wt % of a binder.

In one embodiment, the tablet comprises a binder which is polyvinylpyrrolidone, polyvinylalcohol, ethyl cellulose, or polyethylene glycol. In yet another embodiment, the polyvinylpyrrolidone is povidone, copovidone, or crospovidone. In yet another embodiment, the tablet comprises a combination of more than one binder.

In one embodiment, tablet comprises about 380 mg, 400 mg, 420 mg, 440 mg, 460 gm, 480 mg, 500 mg, 520 mg, 540 mg, 560 mg, 580 mg, 600 mg, or 620 mg of one or more hydrophilic polymers. In another embodiment, the tablet comprises about 15 wt % to about 50 wt % or about 30 wt % to about 50 wt % of a hydrophilic polymer. In yet another embodiment, the tablet comprises about 15 wt %, 18 wt %, 20 wt %, 25 wt %, 28 wt %, 30 wt %, 32 wt %, 33 wt %, 35 wt %, 37 wt %, 40 wt % or 90 wt % of a hydrophilic polymer.

In one embodiment, the tablet comprises one or more hydrophilic polymers, each having an average molecular weight ranging from about 200,000 Da (Daltons) to about 10,000,000 Da, about 900,000 Da to about 5,000,000 Da, about 2,000,000 Da to about 5,000,000 Da, from about 4,000,000 Da to about 5,000,000 Da, from about 2,000,000 Da to about 4,000,000 Da, from about 900,000 Da to about 5,000,000 Da, or from about 900,000 Da to about 4,000,000 Da. In another embodiment, the tablet comprises a hydrophilic polymer having an average molecular weight of about 200,000 Da, 600,000 Da, 900,000 Da, 1,000,000 Da, 2,000,000 Da, 4,000,000 Da, 5,000,000 Da, 7,000,000 Da, or 10,000,000 Da.

In one embodiment, the ER layer comprises a hydrophilic polymer having an average viscosity ranging from about 4,000 cp (centipoise) to about 200,000 cp, from about 50,000 cp to about 200,000 cp, or from about 80,000 cp to about 120,000 cp as measured as a 2% weight per volume in water at 20° C.

In one embodiment, the one or more hydrophilic polymers in the tablet is a polyalkylene oxide. In another embodiment, the hydrophilic polymer is poly(ethylene oxide). In yet another embodiment, the at least one hydrophilic polymer in the tablet is a cellulose. In yet another embodiment, the cellulose is hydroxypropyl methylcellulose.

In one embodiment, the tablet comprises two hydrophilic polymers in a ratio of 1:1, 1.2:1, 1.3:1, 1.4:1, 1.5:1, 1.6:1, 1.8:1, or 2.0:1.

In one embodiment, the tablet comprises about 8 mg, 9 mg, 10 mg, 11 mg, 12 mg, 13 mg, 14 mg, 15 mg of one or more lubricants. In another embodiment, the tablet comprises about 0.5 wt % to about 2.5 wt % of a lubricant. In yet another embodiment, the tablet comprises about 0.5 wt %, 1.0 wt %, 1.5 wt %, 2.0 wt %, or 2.5 wt % of a lubricant.

In one embodiment, the tablet comprises a lubricant which is magnesium stearate, calcium stearate, sodium stearyl fumarate, stearic acid, stearyl behenate, glyceryl behenate, or polyethylene glycol.

In one embodiment, the tablet comprises one or more additional excipients which are diluents, coloring agents, flavoring agents, and/or glidants.

In one embodiment, the dosage form is a single layer tablet. In another embodiment, the dosage form comprises a coat. In yet another embodiment, the coat comprises one or more active agents. In still another embodiment, the coat comprises the azo-bonded prodrug of 5-ASA.

In one embodiment, the dosage form is a bilayer or multilayer tablet.

In one embodiment, the dosage form does not comprise a semipermeable membrane through which the prodrug is released.

In one embodiment, the dosage form comprises a second therapeutic agent. In another embodiment, the second therapeutic agent is an azo-bonded prodrug of 5-ASA. In yet another embodiment, the second therapeutic agent is formulated for immediate release.

In one embodiment, the dosage form is formulated to provide sustained release of the azo-bonded prodrug of 5-ASA and a second therapeutic agent.

In one embodiment, the single layer, bilayer, multilayer tablet has a friability of no greater than about 0.1%, 0.2% 0.3%, 0.4%, 0.5%, 0.7% or 1.0%.

In one embodiment, the tablet has a hardness of at least about 10 kiloponds (kp). In some embodiments, the tablet has a hardness of about 9 kp to about 25 kp, or about 12 kp to about 20 kp. In further embodiments, the tablet has a hardness of about 11 kp, 12 kp, 13 kp, 14 kp, 15 kp, 16 kp, 17 kp, 18 kp, 19 kp, 20 kp, 21 kp, 22 kp, 23 kp, 24 kp or 25 kp.

In one embodiment, the tablets have a content uniformity of from about 85 to about 115 percent by weight or from about 90 to about 110 percent by weight, or from about 95 to about 105 percent by weight. In other embodiments, the content uniformity has a relative standard deviation (RSD) equal to or less than about 3.5%, 3.0%, 2.5%, 2.0%, 1.5%, 1.0% or 0.5%.

In one embodiment, the azo-bonded prodrug of 5-ASA is released from the tablet over a time period of about 5 hours (h) to 13 h, about 6 h to 12 h, about 7 h to 10 h, about 8 h to 9 h, about 6 h to 9 h, about 8 h to 10 h, or about 9 h to 10 h. In another embodiment, the azo-bonded prodrug of 5-ASA is delivered to the colon of a subject over a time period of about 4 h, 5 h, 6 h, 7 h, 8 h, 9 h, 10 h, 11 h, 12 h, or 13 h. In yet another embodiment, the azo-bonded prodrug of 5-ASA is delivered to the colon of a subject over a time period of greater than 12 h.

In one embodiment, the azo-bonded prodrug of 5-ASA is released from the tablet via erosion. In another embodiment, the azo-bonded prodrug of 5-ASA is released from the tablet via diffusion. In yet another embodiment, the azo-bonded prodrug of 5-ASA is released from the tablet via a combination of erosion and diffusion.

In one embodiment, about 5% to about 20% or 10% to 20% of the dose of azo-bonded prodrug of 5-ASA is released within 10 minutes, 15 minutes, 30 minutes, 45 minutes, 60 minutes, 90 minutes or 2 hours after contact with a fluid. In another embodiment, the fluid is water. In still another embodiment, the fluid is gastric fluid or simulated gastric fluid. In another embodiment, the fluid is gastrointestinal fluid. In still another embodiment, the release is measured at about 25° C. to 40° C., 30° C. to 40° C., 35° C. to 40° C., or 20° C. to 30° C.

In one embodiment, not more than about 15%, 20%, 30%, or 40% of the dose of azo-bonded prodrug of 5-ASA is released within about the first hour after contact with fluid. In another embodiment, not more than about 25%, 30%, 35%, 40%, 45%, 50%, 55%, 60%, 65%, 70%, 75%, or 80% of the dose of azo-bonded prodrug of 5-ASA is released within about 4 hours after contact with fluid.

In one embodiment, the tablet swells upon imbibition of fluid from gastric fluid to a size which is at least 15%, 20%, 25%, 30%, 35% 40%, 45%, or 50% larger than the size of the tablet prior to imbibition of fluid. In another embodiment, the tablet swells upon imbibition of fluid from gastric fluid to a size which is approximately 15%, 20%, 25%, 30%, 35% 40%, 45%, or 50% larger than the size of the tablet prior to imbibition of fluid.

In one embodiment, the dosage form provides a dissolution profile wherein between about 50% to about 85%, about 55% to about 80% or about 35% to about 55% of the dose of azo-bonded prodrug of 5-ASA remains in the tablet between about 1 and 2 hours after administration or after contact with a fluid. In yet another embodiment, not less than about 50%, 55%, 60%, 65%, 70%, or 75% is released within about 6 hours after oral administration or contact of the dosage form with a fluid. In yet another embodiment, not less than about 60% is released within about 6 hours after oral administration or contact of the dosage form with a fluid.

In a second aspect, a method of making a gastric retentive dosage form comprising an azo-bonded prodrug of 5-ASA and at least one hydrophilic polymer is provided. In one embodiment, the azo-bonded prodrug of 5-ASA is olsalazine, sulfasalazine or balsalazide, or a pharmaceutically acceptable salt thereof.

In one embodiment, a method of making the dosage form comprising wet granulating azo-bonded prodrug of 5-ASA powder with one or more binders and/or one or more disintegrants is provided, wherein granules are formed. In another embodiment, the method of making the dosage form further comprises screening the dry granules based on size. In yet another embodiment, the method of making the dosage form further comprises blending the granules with additional excipients.

In a third aspect, a gastric retained dosage form comprising an azo-bonded prodrug of 5-ASA and at least one swellable polymer is administered to a subject in need thereof.

In one embodiment, the subject has been diagnosed with an inflammatory bowel disorder. In one embodiment, the inflammatory bowel disorder is ulcerative colitis (UC), inflammatory bowel disorder (IBD) or Crohn's Disease (CD).

In one embodiment, the subject has been diagnosed with an inflammatory arthritis. In another disorder the subject has been diagnosed with rheumatoid arthritis or psoriatic arthritis.

In one embodiment, the subject is administered about 200 mg to 4 g, 100 mg to 1000 mg, 250 mg to 1500 mg, 750 mg to 1500 mg, 500 mg to 1000 mg, 500 mg to 1500 mg, 500 mg to 2 g, 1 g to 2 g, 1 g to 4 g, 1 g to 3 g, 2 g to 4 g, 2 g to 10 g, 4 g to 10 g, 4 g to 6 g, 5 g to 7 g, 6 g to 8 g, or 3 g to 6 g of the azo-bonded prodrug of 5-ASA in a 24 hour period.

In one embodiment, the subject is administered one or more dosage forms, wherein each dosage form comprises about 300 mg to 850 mg, 350 mg to 800 mg, 400 mg to 700 mg, 250 mg to 500 mg, 500 mg to 1000 mg, 700 mg to 1000 mg, 400 mg to 800 mg, 500 mg to 900 mg, 450 mg, 550 mg, 600 mg, 650 mg, 700 mg, 750 mg, 800 mg, or 850 mg of the prodrug.

In one embodiment, a gastric retained dosage form comprising an azo-bonded prodrug of 5-ASA is administered to a subject in a fed mode. In another embodiment, the dosage form is administered with a meal to a subject once in a 24 hour period. In other embodiments, the dosage form is administered with a meal to the subject twice or thrice in a 24 hour period. In yet another embodiment, the dosage form is administered with a meal to a subject once or twice in a 24 hour period for 2, 3, 4, 5, 6, 7, 8 or more days.

In one embodiment, the dosage form is administered to the subject in a fed mode. In another embodiment, the dosage form is administered to the subject with a meal. In still another embodiment, the dosage form is administered to the subject within about 5 min, 10 min, 15 min or 30 min before or after a meal.

In a fourth aspect, a method for treating a subject suffering from an inflammatory bowel disease (IBD) comprising administering a gastric retained azo-bonded prodrug of 5-ASA oral dosage form is provided.

In one embodiment, the azo-bonded prodrug of 5-ASA is olsalazine, sulfasalazine or balsalazide.

In one embodiment, the IBD is ulcerative colitis (UC). In another embodiment the administering reduces bowel inflammation, diarrhea, stool frequency, rectal bleeding, and/or abdominal pain in the subject as compared to the presence of said symptoms in the absence of administration. In still another embodiment, adverse side effects elicited by the administration of the azo-bonded prodrug of 5-ASA are reduced at least about 10%, 15%, 25%, 30%, 40% or 50% as compared to administration of an equivalent dose of an immediate release formulation comprising the azo-bonded prodrug of 5-ASA.

### BRIEF DESCRIPTION OF THE DRAWINGS

FIG. 1 is a graph showing disintegration profiles for tablets containing 500 mg olsalazine (Formulations 1 and 2) at pH 1.2.

FIG. 2 is a graph showing dissolution release profiles for tablets containing 500 mg olsalazine (Formulations 1 and 2) at pH 4.5.

FIG. 3 is a graph showing dissolution release profiles for tablets containing 500 mg olsalazine (Formulations 1 and 2) at pH 7.5.

FIG. 4 is a graph showing dissolution release profiles for tablets containing 750 mg olsalazine (Formulations 3 and 4) at pH 4.5.

FIG. 5 is a graph showing dissolution release profiles for tablets containing 750 mg olsalazine (Formulations 3 and 4) at pH 7.5.

FIG. 6 is a graph showing swelling and erosion of tablets containing 750 mg olsalazine (Formulations 3) at pH 4.5.

FIG. 7 is a graph showing swelling and erosion of tablets containing 750 mg olsalazine (Formulations 3) at pH 7.5.

FIGS. 8-9 are graphs showing swelling profiles for Formulation 2, 3, and 4 tablets at pH 4.5.

FIGS. 10-11 are graphs showing swelling profiles for Formulation 3 and 4 tablets at pH 7.5.

FIG. 12 is a graph showing release of olsalazine from tablets made by using standard olsalazine particle size (triangles and diamonds) and micronized olsalazine particles (diamonds and squares), at pH 7.5 (triangles and diamonds) and pH 4.5 (squares and diamonds).

### DETAILED DESCRIPTION

The various aspects and embodiments will now be fully described herein. These aspects and embodiments may, however, be embodied in many different forms and should not be construed as limiting; rather, these embodiments are provided so the disclosure will be thorough and complete, and will fully convey the scope of the present subject matter to those skilled in the art.

### I. DEFINITIONS

It must be noted that, as used in this specification, the singular forms “a,” “an,” and “the” include plural referents unless the context clearly dictates otherwise.

Compounds useful in the compositions and methods include those described herein in any of their pharmaceutically acceptable forms, including isomers such as diastereomers and enantiomers, salts, solvates, and polymorphs, as well as racemic mixtures and pure isomers of the compounds described herein, where applicable.

“Optional” or “optionally,” as used herein, means that the subsequently described element, component or circumstance may or may not occur, so that the description includes instances where the element, component, or circumstance occurs and instances where it does not.

The terms “subject,” “individual” or “patient” are used interchangeably herein and refer to a vertebrate, preferably a mammal. Mammals include, but are not limited to, humans.

The term “about”, particularly in reference to a given quantity, is meant to encompass deviations of plus or minus five percent.

The term “fed mode,” as used herein, refers to a state which is typically induced in a patient by the presence of food in the stomach, the food-giving rise to two signals, one that is said to stem from stomach distension and the other a chemical signal based on food in the stomach. It has been determined that once the fed mode has been induced, larger particles are retained in the stomach for a longer period of time than smaller particles; thus, the fed mode is typically induced in a patient by the presence of food in the stomach. The fed mode is initiated by nutritive materials entering the stomach upon the ingestion of food. Initiation is accompanied by a rapid and profound change in the motor pattern of the upper GI tract, over a period of 30 seconds to one minute. The change is observed almost simultaneously at all sites along the G.I. tract and occurs before the stomach contents have reached the distal small intestine. Once the fed mode is established, the stomach generates 3-4 continuous and regular contractions per minute, similar to those of the fasting mode but with about a quarter to half the amplitude (Force). The pylorus is partially open, causing a sieving effect in which liquids and small particles flow continuously from the stomach into the intestine while indigestible particles greater in size than the pyloric opening are retropelled and retained in the stomach. This sieving effect thus causes the stomach to retain particles exceeding about 1 cm in size for approximately 4 to 8 hours or more. Administration of a dosage form “with a meal,” as used herein, refers to administration during or after a meal. When the dosage form is administered after a meal, it may be administered about 1, 2, 3, 4, 5, 10, 15 minutes after completion of a meal.

A drug “release rate,” as used herein, refers to the quantity of drug released from a dosage form or pharmaceutical composition per unit time, e.g., milligrams of drug released per hour (mg/hr). Drug release rates for drug dosage forms are typically measured as an in vitro rate of dissolution, i.e., a quantity of drug released from the dosage form or pharmaceutical composition per unit time measured under appropriate conditions and in a suitable fluid. The specific results of drug release tests claimed herein were obtained for the dosage forms or pharmaceutical compositions using a USP Dissolution apparatus III (APP III) or a Disintegration tester. Tests were performed at about pH 1.2 (modified simulated gastric fluid, or mSFG), pH 4.5 (the approximate pH of the stomach after a meal) or at pH 7.5, at 37° C. Suitable aliquots of the release rate solutions are tested to determine the amount of drug released from the dosage form or pharmaceutical composition. For example, the drug can be assayed or injected into a chromatographic system to quantify the amounts of drug released during the testing intervals. The release rate is sometimes measured beginning at the time at which the dosage form contacts a fluid such as water, gastric fluid or simulated gastric fluid. The fluid can be formulated to have a pH which simulates the pH of the stomach, small intestine and/or large intestine. This release rate is considered to represent the rate of release after oral administration of the dosage form.

The terms “hydrophilic” and “hydrophobic” are generally defined in terms of a partition coefficient P, which is the ratio of the equilibrium concentration of a compound in an organic phase to that in an aqueous phase. A hydrophilic compound has a P value less than 1.0, typically less than about 0.5, where P is the partition coefficient of the compound between octanol and water, while hydrophobic compounds will generally have a P greater than about 1.0, typically greater than about 5.0. The polymeric carriers herein are hydrophilic, and thus compatible with aqueous fluids such as those present in the human body.

The term “polymer” as used herein refers to a molecule containing a plurality of covalently attached monomer units, and includes branched, dendrimeric, and star polymers as well as linear polymers. The term also includes both homopolymers and copolymers, e.g., random copolymers, block copolymers and graft copolymers, as well as uncrosslinked polymers and slightly to moderately to substantially crosslinked polymers, as well as two or more interpenetrating cross-linked networks.

The term “swellable polymer,” as used herein, refers to a polymer that will swell in the presence of a fluid. It is understood that a given polymer may or may not swell when present in a defined drug formulation. Accordingly, the term “swellable polymer” defines a structural feature of a polymer which is dependent upon the composition in which the polymer is formulated. Whether or not a polymer swells in the presence of fluid will depend upon a variety of factors, including the specific type of polymer and the percentage of that polymer in a particular formulation. For example, the term “polyethylene oxide” or “PEO” refers to a polyethylene oxide polymer that has a wide range of molecular weights. PEO is a linear polymer of unsubstituted ethylene oxide and has a wide range of viscosity-average molecular weights. Examples of commercially available PEOs and their approximate molecular weights are: POLYOX® NF, grade WSR coagulant, approximate molecular weight 5 million, POLYOX® grade WSR 301, approximate molecular weight 4 million, POLYOX® grade WSR 303, approximate molecular weight 7 million, POLYOX®grade WSR N-60K, approximate molecular weight 2 million, and POLYOX® grade N-80K, approximate molecular weight 200,000. It will be understood by a person with ordinary skill in the art that an oral dosage form which comprises a swellable polymer will swell upon imbibition of water or fluid from gastric fluid.

The terms “swellable” and “bioerodible” (or simply “erodible”) are used to refer to the polymers used in the present dosage forms, with “swellable” polymers being those that are capable of absorbing water and physically swelling as a result, with the extent to which a polymer can swell being determined by the molecular weight or degree of crosslinking (for crosslinked polymers), and “bioerodible” or “erodible” polymers referring to polymers that slowly dissolve and/or gradually hydrolyze in an aqueous fluid, and/or that physically disentangle or undergo chemical degradation of the chains themselves, as a result of movement within the stomach or GI tract.

The term “friability,” as used herein, refers to the ease with which a tablet will break or fracture. The test for friability is a standard test known to one skilled in the art. Friability is measured under standardized conditions by weighing out a certain number of tablets (generally 20 tablets or less), placing them in a rotating Plexiglas drum in which they are lifted during replicate revolutions by a radial lever, and then dropped approximately 8 inches. After replicate revolutions (typically 100 revolutions at 25 rpm), the tablets are reweighed and the percentage of formulation abraded or chipped is calculated. The friability of the tablets, of the present invention, is preferably in the range of about 0% to 3%, and values about 1%, or less, are considered acceptable for most drug and food tablet contexts. Friability which approaches 0% is particularly preferred.

The term “tap density” or “tapped density,” as used herein, refers to a measure of the density of a powder. The tapped density of a pharmaceutical powder is determined using a tapped density tester, which is set to tap the powder at a fixed impact force and frequency. Tapped density by the USP method is determined by a linear progression of the number of taps.

The term “bulk density,” as used herein, refers to a property of powders and is defined as the mass of many particles of the material divided by the total volume they occupy. The total volume includes particle volume, inter-particle void volume and internal pore volume.

The term “capping,” as used herein, refers to the partial or complete separation of top or bottom crowns of the tablet main body. For multilayer tablets, capping refers to separation of a portion of an individual layer within the multilayer tablet. Unintended separation of layers within a multilayer tablet prior to administration is referred to herein as “splitting.”

The term “content uniformity,” as used herein refers to the testing of compressed tablets to provide an assessment of how uniformly the micronized or submicron active ingredient is dispersed in the powder mixture. Content uniformity is measured by use of USP Method (General Chapters, Uniformity of Dosage Forms), unless otherwise indicated. A plurality refers to five, ten or more tablet compositions.

The terms “effective amount” or a “therapeutically effective amount” refer to the amount of drug or pharmacologically active agent to provide the desired effect without toxic effects. The amount of an agent that is “effective” may vary from individual to individual, depending on the age, weight, general condition, and other factors of the individual. An appropriate “effective” amount in any individual may be determined by one of ordinary skill in the art using routine experimentation. An “effective amount” of an agent can refer to an amount that is either therapeutically effective or prophylactically effective or both.

By “pharmaceutically acceptable,” such as in the recitation of a “pharmaceutically acceptable carrier,” or a “pharmaceutically acceptable acid addition salt,” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. The term “pharmacologically active” (or simply “active”) as in a “pharmacologically active” derivative, refers to a derivative having the same type of pharmacological activity as the parent compound and/or drug and approximately equivalent in degree. When the term “pharmaceutically acceptable” is used to refer to a derivative (e.g., a salt) of an active agent, it is to be understood that the compound is pharmacologically active as well. When the term, “pharmaceutically acceptable” is used to refer to an excipient, it implies that the excipient has met the required standards of toxicological and manufacturing testing or that it is on the Inactive Ingredient Guide prepared by the FDA, or comparable agency.

The terms “drug,” “active agent,” “therapeutic agent,” and/or “pharmacologically active agent” are used interchangeably herein to refer to any chemical compound, complex or composition that is suitable for oral administration and that has a beneficial biological effect, preferably a therapeutic effect in the treatment or prevention of a disease or abnormal physiological condition. The terms also encompass pharmaceutically acceptable, pharmacologically active derivatives of those active agents specifically mentioned herein, including, but not limited to, salts, esters, amides, prodrugs, active metabolites, analogs, and the like. When the terms “active agent,” “pharmacologically active agent,” and “drug” are used, then, or when a particular active agent is specifically identified, it is to be understood that applicants intend to include the active agent per se as well as pharmaceutically acceptable, pharmacologically active salts, esters, amides, prodrugs, metabolites, analogs, etc.

The term “dosage form” refers to the physical formulation of the drug for administration to the patient. Dosage forms include without limitation, tablets, capsules, caplets, liquids, syrups, lotions, lozenges, aerosols, patches, enemas, oils, ointments, pastes, powders for reconstitution, sachets, solutions, sponges, and wipes. Within the context of the present invention, a dosage form comprising an azo-bonded prodrug of 5-ASA formulation will generally be administered to patients in the form of tablets.

The term “dosage unit” refers to a single unit of the dosage form that is to be administered to the patient. The dosage unit will be typically formulated to include an amount of drug sufficient to achieve a therapeutic effect with a single administration of the dosage unit although where the size of the dosage form is at issue, more than one dosage unit may be necessary to achieve the desired therapeutic effect. For example, a single dosage unit of a drug is typically, one tablet, one capsule, or one tablespoon of liquid. More than one dosage unit may be necessary to administer sufficient drug to achieve a therapeutic effect where the amount of drug causes physical constraints on the size of the dosage form.

“Delayed release” dosage forms are a category of modified release dosage forms in which the release of the drug is delayed after oral administration for a finite period of time after which release of the drug is unhindered. Delayed release dosage forms are frequently used to protect an acid-labile drug from the low pH of the stomach or where appropriate to target the GI tract for local effect while minimizing systemic exposure. Enteric coating is frequently used to manufacture delayed release dosage forms.

The terms “sustained release,” and “extended release” are used interchangeably herein to refer to a dosage form that provides for gradual release of a drug over an extended period of time. With extended release dosage forms, the rate of release of the drug from the dosage form is reduced in order to maintain therapeutic activity of the drug for a longer period of time or to reduce any toxic effects associated with a particular dosing of the drug. Extended release dosage forms have the advantage of providing patients with a dosing regimen that allows for less frequent dosing, thus enhancing compliance. Extended release dosage forms can also reduce peak-related side effects associated with some drugs and can maintain therapeutic concentrations throughout the dosing period thus avoiding periods of insufficient therapeutic plasma concentrations between doses.

The term “modified release” refers to a dosage form that includes both delayed and extended release drug products. The manufacture of delayed, extended, and modified release dosage forms are known to ordinary skill in the art and include the formulation of the dosage forms with excipients or combinations of excipients necessary to produce the desired active agent release profile for the dosage form.

The “gastric retentive” oral dosage forms described herein are a type of extended release dosage form. Gastric retentive dosage forms are beneficial for the delivery of drugs with reduced absorption in the lower GI tract or for local treatment of diseases of the stomach or upper GI tract. For example, in certain embodiments of gastric retentive oral dosage forms of the present invention, the dosage form swells in the gastric cavity and is retained in the gastric cavity of a patient in the fed med so that the drug may be released for heightened therapeutic effect. See, Hou et al., Crit. Rev. Ther. Drug Carrier Syst. 20(6):459-497 (2003).

The in vivo “release rate” and in vivo “release profile” refer to the time it takes for the orally administered dosage form, or the active agent-containing layer of a bilayer or multilayer tablet (administered when the stomach is in the fed mode) or the content of the active ingredient to be reduced to 0-10%, preferably 0-5%, of its original size or level, as may be observed visually using NMR shift reagents or paramagnetic species, radio-opaque species or markers, or radiolabels, or determined mathematically, such as deconvolution, upon its plasma concentration profiles.

The term “AUC” (i.e., “area under the curve,” “area under the concentration curve,” or “area under the concentration-time curve”) is a pharmacokinetic term used to refer a method of measurement of bioavailability or extent of absorption of a drug based on a plot of an individual or pool of individual's blood plasma concentrations sampled at frequent intervals; the AUC is directly proportional to the total amount of unaltered drug in the patient's blood plasma. For example, a linear curve for a plot of the AUC versus dose (i.e., straight ascending line) indicates that the drug is being released slowly into the blood stream and is providing a steady amount of drug to the patient; if the AUC versus dose is a linear relationship this generally represents optimal delivery of the drug into the patient's blood stream. By contrast, a non-linear AUC versus dose curve indicates rapid release of drug such that some of the drug is not absorbed, or the drug is metabolized before entering the blood stream.

The term “Cmax” (i.e., “maximum concentration”) is a pharmacokinetic term used to indicate the peak concentration of a particular drug in the blood plasma of a patient.

The term “Tmax” (i.e., “time of maximum concentration” or “time of Cmax”) is a pharmacokinetic term used to indicate the time at which the Cmax is observed during the time course of a drug administration. As would be expected, a dosage form that would include an immediate release as well as a gastric retentive component would have a Tmax that is higher than the Cmax for an immediate release dosage form, but lower than the Tmax for a purely gastric retentive dosage form.

“Preventing,” in reference to a disorder or unwanted physiological event in a patient, refers specifically to inhibiting or significant reducing the occurrence of symptoms associated with the disorder and/or the underlying cause of the symptoms.

“Therapeutically effective amount,” in reference to a therapeutic agent, refers to an amount that is effective to achieve a desired therapeutic result. Therapeutically effective amounts of a given agent will typically vary with respect to factors such as the type and severity of the disorder or disease being treated and the age, gender, weight and other factors of the patient.

“Treating,” “treat,” and “treatment” refer to reduction in severity and/or frequency of symptoms, elimination of symptoms and/or underlying cause, prevention of the occurrence of symptoms and/or their underlying cause, and improvement or remediation of damage.

### II. GASTRIC RETENTIVE DOSAGE FORM FOR THE EXTENDED RELEASE OF AN AZO-BONDED PRODRUG OF 5-ASA

The pharmaceutical compositions described herein, i.e., gastric retained dosage forms comprising an azo-bonded prodrug of 5-ASA such as olsalazine, sulfasalazine or balsalazide, provide extended or sustained release of the azo-bonded prodrug of 5-ASA to the upper gastrointestinal tract. The presently described dosage forms provide for extended release of the azo-bonded prodrug of 5-ASA in the stomach wherein the dosage forms are comprised of a polymer matrix that swells upon imbibition of fluid to a size sufficient for gastric retention. Thus, in formulating the dosage forms, it is desirable to provide the properties which simultaneously allow: a) an extent of swelling to provide gastric retention over an extended period, and b) a rate of swelling and erosion that allows release of the azo-bonded prodrug of 5-ASA over a time period of approximately 6 to 12 hours.

Olsalazine, sulfasalazine and balsalazide are exemplary prodrugs which are each converted to the active agent, 5-ASA, only in the colon. 5-ASA is an anti-inflammation drug which acts topically to reduce and/or prevent inflammation of the colonic tissue. Currently marketed formulations of azo-bonded prodrugs of 5-ASA are designed for immediate release. Accordingly, in the case of olsalazine, the bolus of olsalazine delivered to the GI tract causes a secretory response in the ileum resulting in increased diarrhea, as well as higher concentrations of 5-ASA in the colon such that there is increased unwanted systemic absorption. This same effect occurs to a lesser extent with sulfasalazine and balsalazide dosage forms.

Described herein are oral dosage forms that provide sustained release of an azo-bonded prodrug of 5-ASA into the stomach for at least 8-12 hours, thereby resulting in continual bathing of the colon in 5-ASA present at concentrations which are therapeutically effective, but lower than concentrations provided by immediate release formulations of an azo-bonded prodrug of 5-ASA. Sustained release gastric retained dosage forms containing an azo-bonded prodrug of 5-ASA as presented below overcome many of the limitations of the currently available prodrug dosage formulations.

Moreover, the formulation of these pharmaceutical oral dosage forms must result in final products that meet the requirements of regulatory agencies such as the Food and Drug Administration. For example, final products must have a stable product that does not fracture during storage and transport. This is measured for tablets, in part, in terms of friability and hardness. Dosage forms must also meet the requirements for content uniformity, which essentially means that the dispersion of the active ingredient(s) is uniform throughout the mixture used to make the dosage form, such that the composition of tablets formed from a particular formulation does not vary significantly from one tablet to another. The FDA requires a content uniformity within a range of 95% to 105%.

The dosage form as described here comprises one or more swellable polymers and is capable of swelling dimensionally unrestrained in the stomach upon contact with gastric fluid due to the component hydrophilic polymers, for example, polyethylene oxide and/or hypromellose (also known as hydroxypropyl methylcellulose or HPMC), and increase to a size sufficient to be retained in the stomach in a fed mode.

Water-swellable polymers suitable for use herein are those that swell in a dimensionally unrestrained manner upon contact with water Such polymers may also gradually erode over time. Examples of such polymers include polyalkylene oxides, such as polyethylene glycols, particularly high molecular weight polyethylene glycols; cellulose polymers and their derivatives including, but not limited to, hydroxyalkyl celluloses, hydroxymethyl cellulose, hydroxyethyl cellulose, hydroxypropyl cellulose, hydroxypropylmethyl cellulose, carboxymethylcellulose, microcrystalline cellulose; polysaccharides and their derivatives; chitosan; poly(vinyl alcohol); xanthan gum; maleic anhydride copolymers; poly(vinyl pyrrolidone); starch and starch-based polymers; maltodextrins; poly(2-ethyl-2-oxazoline); poly(ethyleneimine); polyurethane; hydrogels; crosslinked polyacrylic acids; and combinations or blends of any of the foregoing.

Further examples are copolymers, including block copolymers and graft polymers. Specific examples of copolymers are PLURONIC® and TECTONIC®, which are polyethylene oxide-polypropylene oxide block copolymers available from BASF Corporation, Chemicals Div., Wyandotte, Mich., USA. Further examples are hydrolyzed starch polyacrylonitrile graft copolymers, commonly known as “Super Slurper” and available from Illinois Corn Growers Association, Bloomington, Ill., USA.

Preferred swellable, erodible hydrophilic polymers suitable for forming the gastric retentive portion of the dosage forms described herein are poly(ethylene oxide), hydroxypropyl methyl cellulose, and combinations of poly(ethylene oxide) and hydroxypropyl methyl cellulose. Poly(ethylene oxide) is used herein to refer to a linear polymer of unsubstituted ethylene oxide. The molecular weight of the poly(ethylene oxide) polymers can range from about 9×10⁵ Daltons to about 8×10⁶ Daltons. Exemplary molecular weight poly(ethylene oxide) polymers include about 9×10⁵ Daltons (e.g., SENTRY™ POLYOX™ WSR 1105, NF Grade, Dow Chemical), 2×10⁶ Daltons (e.g., SENTRY™ POLYOX™ WSR N60K, NF Grade, Dow Chemical), 2×10⁶ Daltons (e.g., SENTRY™ POLYOX™ WSR 301, NF Grade, Dow Chemical), and 7×10⁶ Daltons (e.g., SENTRY™ POLYOX™ WSR N60K, NF Grade, Dow Chemical). The viscosity of a 1% water solution of the polymer at 25° C. preferably ranges from 4500 to 7500 centipoise.

Dosage forms prepared for oral administration according to the present disclosure will generally contain other inactive additives (excipients) such as binders, lubricants, disintegrants, fillers, stabilizers, surfactants, coloring agents, and the like.

Binders are used to impart cohesive qualities to a tablet, and thus ensure that the tablet or tablet layer remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Examples of polyvinylpyrrolidone include povidone, copovidone and crospovidone.

Lubricants are used to facilitate tablet manufacture, promoting powder flow and preventing particle capping (i.e., particle breakage) when pressure is relieved. Useful lubricants are magnesium stearate (in a concentration of from 0.25 wt % to 3 wt %, 0.2 wt % to 1.5 wt %, or about 1.0 wt %), calcium stearate, stearic acid, and hydrogenated vegetable oil (e.g., comprised of hydrogenated and refined triglycerides of stearic and palmitic acids at about 1 wt % to 5 wt % or less than about 2 wt %). Disintegrants are used to facilitate disintegration of the tablet, thereby increasing the erosion rate relative to the dissolution rate, and are generally starches, clays, celluloses, algins, gums, or crosslinked polymers (e.g., crosslinked polyvinyl pyrrolidone). Fillers include, for example, materials such as silicon dioxide, titanium dioxide, alumina, talc, kaolin, powdered cellulose, and microcrystalline cellulose, as well as soluble materials such as mannitol, urea, sucrose, lactose, lactose monohydrate, dextrose, sodium chloride, and sorbitol. Solubility-enhancers, including solubilizers per se, emulsifiers, and complexing agents (e.g., cyclodextrins), may also be advantageously included in the present formulations. Stabilizers, as well known in the art, are used to inhibit or retard drug decomposition reactions that include, by way of example, oxidative reactions.

The dosage form may further comprise a chelating agent. Examples of chelating agents include ethylenediamine tetracetic acid (EDTA) and its salts (including a sodium salt), N-(hydroxy-ethyl)ethylenediaminetriacetic acid, nitrilotriacetic acid (NIA), ethylene-bis(oxyethylene-nitrilo)tetraacetic acid, 1,4,7,10-tetraazacyclodo-decane-N,N′,N″,N′″-tetraacetic acid, 1,4,7,10-tetraaza-cyclododecane-N,N′,N″-triacetic acid, 1,4,7-tris(carboxymethyl)-10-(2′-hydroxypropyl)-1,4,7,10-tetraazocyclodecane, 1,4,7-triazacyclonane-N,N′,N″-triacetic acid, 1,4,8,11-tetraazacyclotetra-decane-N,N′,N″,N′″-tetraacetic acid; diethylenetriamine-pentaacetic acid (DTPA), ethylenedicysteine, bis(aminoethanethiol)carboxylic acid, triethylenetetraamine-hexaacetic acid, and 1,2-diaminocyclohexane-N,N,N′,N′-tetraacetic acid. The chelating agent may be present in the dosage form in an amount that is about 0.01 wt % to about 0.10 wt % or about 0.02 to about 0.08 wt % of the tablet. Alternatively, the table may comprise about 0.01 wt %, 0.02 wt %, 0.03 wt %, 0.04 wt %, 0.05 wt %, 0.06 wt %, 0.07 wt %, 0.08 wt %, 0.09 wt % or 0.10 wt % of the chelating agent.

The gastric retentive dosage form may be a single layer, bilayer, or multilayer tablet or it may be a capsule. Multilayer tablets include tablets having a shell-and-core configuration in which a core is fully encased by a shell. Tablets may also have a coating with or without the pharmaceutically active agent. The tablet comprises a gastric retentive layer which comprises an azo-bonded prodrug of 5-ASA dispersed in a matrix comprised of at least one hydrophilic polymer which swells upon imbibition of fluid.

In one embodiment, a dosage form is formulated to have a dual-matrix configuration (“shell-and-core”) as described in U.S. Patent Publication No. 2003/0104062 (herein incorporated by reference). One matrix forms a core of polymeric material in which the azo-bonded prodrug of 5-ASA is dispersed and the other matrix forms a casing that surrounds and fully encases the core, the casing being of polymeric material that swells upon imbibition of water (and hence gastric fluid) to a size large enough to promote retention in the stomach during the fed mode, the shell and core being configured such that the drug contained in the core is released from the dosage form by diffusion through the shell. The shell is sufficient thickness and strength that it is not disrupted by the swelling and remains intact during substantially the entire period of drug release. The shell may or may not contain the azo-bonded prodrug of 5-ASA.

Water-swellable polymers useful in the preparation of the shell-and-core dosage form include polymers that are non-toxic and, at least in the case of the shell, polymers that swell in a dimensionally unrestricted manner upon imbibition of water. The core polymer may also be a swelling polymer, and if so, compatible polymers will be selected that will swell together without disrupting the integrity of the shell. The core and shell polymers may be the same or different, and if the same, they may vary in molecular weight, crosslinking density, copolymer ratio, or any other parameter that affects the swelling rate, so long as any swelling occurring in the core causes substantially not splitting of the shell.

In one embodiment, a tablet having an immediate release layer encased by an extended release gastric retained layer as a shell is manufactured. The shell-and-core tablet can then be spray-coated with an IR layer.

In one embodiment, a tablet is formulated to have an extended release gastric retained (GR) layer spray-coated with an immediate release (IR layer) to provide 12-hour release of an azo-bonded prodrug of 5-ASA. In a further embodiment, the tablet contains 750 mg azo-bonded prodrug of 5-ASA, with 75 mg, 100 mg, 125 mg, or 150 mg azo-bonded prodrug of 5-ASA in the IR layer and 675 mg, 650 mg, 625 mg, or 600 mg azo-bonded prodrug of 5-ASA, respectively, in the GR layer.

In one embodiment, an oral dosage form is formulated as a pulsatile release dosage form, as described in U.S. Patent Publication No. 2009/028941 (incorporated herein by reference). In this embodiment, the oral dosage form may be comprised of: 1) a plurality of immediate release beads containing an azo-bonded prodrug of 5-ASA, dispersed in a hydrophilic polymer that swells unrestrained dimensionally in water; 2) a series of inserts, each containing a plurality of immediate release beads containing an azo-bonded prodrug of 5-ASA, and each comprised of a swellable hydrophilic polymeric matrix, wherein the inserts are stacked to provide pulses of prodrug over an extended period of time; and 3) a swellable hydrophilic matrix comprised of two or more regions, each region containing a dose of azo-bonded prodrug of 5-ASA, wherein the regions vary in size and position within the matrix to effect pulsed release of the azo-bonded prodrug of 5-ASA over an extended period of time.

### III. METHODS FOR MAKING THE DOSAGE FORMS

The presently described dosage forms provide for extended release of an azo-bonded prodrug of 5-ASA in the stomach wherein the dosage forms are comprised of a polymer matrix that swells upon imbibition of fluid to a size sufficient for gastric retention. Thus, in formulating the dosage forms, it is desirable to provide the properties which simultaneously allow: a) an extent of swelling to provide gastric retention over an extended period, and b) a rate of swelling and erosion that allows release of the azo-bonded prodrug of 5-ASA over a time period of approximately 8 to 12 hours.

Moreover, the formulation of these pharmaceutical oral dosage forms preferably result in final products that meet the requirements of regulatory agencies such as the Food and Drug Administration. For example, final products desirably have a stable product that does not fracture during storage and transport. This is measured for tablets, in part, in terms of friability and hardness. Dosage forms preferably also satisfy requirements for content uniformity, which essentially means that the dispersion of the active ingredient(s) is uniform throughout the mixture used to make the dosage form, such that the composition of tablets formed from a particular formulation does not vary significantly from one tablet to another. The FDA requires a content uniformity within a range of 95% to 105%.

The ability to formulate a pharmaceutical oral dosage form which both delivers the therapeutically effective ingredient over a desired period of time and meets FDA requirements depends, in part and in some embodiments, upon the process by which the product is made.

In the case of gastric retentive tablets containing an azo-bonded prodrug of 5-ASA, as disclosed herein, tablets may be made through direct compression or following a granulation procedure. Direct compression is used with a group of ingredients can be blended, placed onto a tablet press, and made into a perfect tablet without any of the ingredients having to be changed. Powders that can be blended and compressed are commonly referred to as directly compressible or as direct-blend formulations. When powders do not compress correctly, a granulation technique is considered.

Granulation is a manufacturing process which increases the size and homogeneity of active pharmaceutical ingredients and excipients which comprise a solid dose formulation. The granulation process, which is often referred to as agglomeration, changes physical characteristics of the dry formulation, with the aim of improving manufacturability, and therefore, product quality.

Granulation technology can be classified into one of two basic types: wet granulation and dry granulation. Wet granulation is by far the more prevalent agglomeration process utilized within the pharmaceutical industry. Most wet granulation procedures follow some basic steps; the drug(s) and excipients are mixed together, and a binder solution is prepared and added to the powder mixture to form a wet mass. The moist particles are then dried and sized by milling or by screening through a sieve. In some cases, the wet granulation is “wet milled” or sized through screens before the drying step. There are four basic types of wet granulation; high shear granulation, fluid bed granulation, extrusion and spheronization and spray drying.

A. Dry Granulation

The dry granulation process involves three basic steps; the drug(s) and excipients(s) are mixed (along with a suitable binder if needed) and some form of lubrication, the powder mixture is compressed into dry “compacts,” and then the compacts are sized by a milling step. The two methods by which dry granulation can be accomplished are slugging and roller compaction.

B. Fluid Bed (Wet) Granulation

The fluid bed granulation process involves the suspension of particulates within an air stream while a granulation solution is sprayed down onto the fluidized bed. During the process, the particles are gradually wetted as they pass through the spay zone, where they become tacky as a result of the moisture and the presence of binder within the spray solution. These wetted particles come into contact with, and adhere to, other wetted particles resulting in the formation of particles.

A fluid bed granulator consists of a product container into which the dry powders are charged, an expansion chamber which sits directly on top of the product container, a spray gun assembly, which protrudes through the expansion chamber and is directed down onto the product bed, and air handling equipment positioned upstream and downstream from the processing chamber.

The fluidized bed is maintained by a downstream blower which creates negative pressure within the product container/expansion chamber by pulling air through the system. Upstream, the air is “pre-conditioned” to target values for humidity, temperature and dew point, while special product retention screens and filters keep the powder within the fluid bed system.

As the air is drawn through the product retention screen it “lifts” the powder out of the product container and into the expansion chamber. Since the diameter of the expansion chamber is greater than that of the product container, the air velocity becomes lower within the expansion chamber. This design allows for a higher velocity of air to fluidize the powder bed causing the material to enter the spray zone where granulation occurs before loosing velocity and falling back down into the product container. This cycle continues throughout the granulation process.

The fluid bed granulation process can be characterized as having three distinct phases; pre-conditioning, granulation and drying. In the initial phase, the process air is pre-conditioned to achieve target values for temperature and humidity, while by-passing the product container altogether. Once desired or optimal conditions are met, the process air is re-directed to flow through the product container, and the process air volume is adjusted to a level which will maintain sufficient fluidization of the powder bed. This pre-conditioning phase completes when the product bed temperature is within the target range specified for the process.

In the next phase of the process, the spraying of the granulating solution begins. The spray rate is set to a fall within a pre-determined range, and the process continues until all of the solution has been sprayed into the batch. It is in this phase where the actual granulation, or agglomeration, takes place.

Once the binder solution is exhausted, the product continues to be fluidized with warm process air until the desired end-point for moisture content is reached. This end-point often correlates well with product bed temperature, therefore in a manufacturing environment, the process can usually be terminated once the target product bed temperature is reached. A typical fluid bed process may require only about thirty to forty-five minutes for the granulation step, plus ten to fifteen minutes on either side for pre-conditioning and drying.

As with any of the wet granulation processes, a variable is the amount of moisture required to achieve successful agglomeration. The fluid bed granulation process preferably provides a “thermodynamic” balance between process air temperature, process air humidity, process air volume and granulation spray rate. While higher process air temperature and process air volume add more heat to the system and remove moisture, more granulating solution and a higher solution spray rate add moisture and remove heat via evaporative cooling. These are process parameters which are preferably evaluated as a manufacturing process is developed, and a key is understanding the interdependency of each variable.

Additional factors affecting the outcome of the fluid bed granulation process are the amount and type of binder solution, and the method by which the binder is incorporated within the granulation. Other process variables are the total amount of moisture added through the process, and the rate at which the moisture content is increased. These parameters can have an effect on the quality and the characteristics of the granulation. For instance, a wetter fluid bed granulation process tends to result in a stronger granule with a higher bulk density. However, an overly aggressive process, where moisture is added too rapidly, can loose control over achieving the final particle size and particle size distribution objectives.

C. High Shear Granulation

Most pharmaceutical products manufactured by wet granulation utilize a high shear process, where blending and wet massing are accomplished by the mechanical energy generated by an impeller and a chopper. Mixing, densification and agglomeration are achieved through the “shear” forces exerted by the impeller; hence the process is referred to as high shear granulation.

The process begins by adding the dry powders of the formulation to the high shear granulator, which is a sealed “mixing bowl” with an impellor which rotates through the powder bed, and a chopper blade which breaks up over-agglomerates which can form during the process. There are typically three phases to the high shear process; dry mixing, solution addition, or wet massing and high shear granulation.

In the first phase, dry powders are mixed together by the impeller blade which rotates through the powder bed. The impeller blade is positioned just off the bottom of the product container. There is a similar tolerance between the tips of the impeller blade and the sides of the container. The impeller blades rotation trough the powder bed creates a “roping” vortex of powder movement. The dry mixing phase typically lasts for only a few minutes.

In the second phase of the process, a granulating liquid is added to the sealed product container, usually by use of a peristaltic pump. The solution most often contains a binder with sufficient viscosity to cause the wet massed particles to stick together or agglomerate. It is common for the solution addition phase to last over a period of from three to five minutes. While the impeller is rotating rather slowly during this step of the process, the chopper blade is turning at a fairly high rate of speed, and is positioned within the product container to chop up over-sized agglomerates, while not interfering with the impellers movement.

Once the binder solution has been added to the product container, the final stage of the granulation process begins. In this phase, high shear forces are generated as the impeller blades push through the wet massed powder bed, further distributing the binder and intimately mixing the ingredients contained therein. The impeller and chopper tool continue to rotate until the process is discontinued when the desired granule particle size and density end-points are reached. This end-point is often determined by the power consumption and/or torque on the impeller.

Once the high shear granulation process has been completed, the material is transferred to a fluid bed dryer, or alternatively, spread out onto trays which are then placed in a drying oven, where the product is dried until the desired moisture content is achieved, usually on the order of 1-2% as measured by Loss On Drying technique.

A variable which affects the high shear process is the amount of moisture required to achieve a successful granulation. A key to the process is having the right amount of moisture to allow for agglomeration to occur. Too little moisture will result in an under-granulated batch, with weak bonds between particles and smaller, to non-existent particles, with properties similar to those of the dry powder starting materials. On the other hand, excess moisture can result in a “crashed” batch with results varying from severe over-agglomeration to a batch which appears more like soup.

Other formulation parameters affecting the outcome of the high shear granulation process are the amount and type of binder solution, and the method by which the binder is incorporated within the granulation. For example, it is possible to include some of the binder in the dry powder mixture as well as in the granulating solution, or it may be incorporated only in the granulating solution or only in the dry powder, as is the case where water is used as the granulating solution.

The high shear granulation process parameters which are variable include impeller and chopper speeds, the solution addition rate, and the amount of time allocated to the various phases of the process. Of these, variables for consideration are the solution addition rate and the amount of time the wet massed product is under high shear mixing

D. Extrusion and Spheronization

This specialized wet granulation technique involves multiple processing steps and was developed to produce very uniform, spherical particles ideally suited for multi-particulate drug delivery of delayed and sustained release dosage forms.

Similar to high shear granulation initially, the first step involves the mixing and wet massing of the formulation. Once this step is complete, the wet particles are transferred to an extruder which generates very high forces used to press the material out through small holes in the extruder head. The extrudate is of uniform diameter and is then transferred onto a rotating plate for spheronization. The forces generated by the rotating plate initially break up the extruded formulation strands into uniform lengths. Additional dwell time within the spheronizer creates particles which are quite round and very uniform in size. These pellets or spheres must then be dried to the target moisture content, usually within a fluid bed system.

Particles produced in this manner tend to be very dense, and have a capacity for high drug loading, approaching 90% or more in some cases. Preferably, particle size is uniform, and the size distribution is narrow, as compared to other granulation approaches. This quality assures consistent surface area within and between batches, which is desired when functional coatings are subsequently applied to create sustained release formulations, delayed release formulations and formulations designed to target a specific area within the body.

Uniform surface area is desired because the pharmaceutical coating process endpoint is determined not by coating thickness, but by the theoretical batch weight gain of the coating material. If the batch surface area is consistent, then the coating thickness will also be consistent for a given weight gain, and coating thickness is the primary variable in determining the functionality of the coating system, whether the goal is controlling the duration of sustained release formulations or imparting an acid resistant characteristic to “beads” necessary to protect certain compounds which would otherwise be severely degraded in the presence of the acidic environment of the stomach.

E. Spray Drying

Spray drying is a unique and specialized process which converts liquids into dry powders. The process involves the spraying of very finely atomized droplets of solution into a “bed” or stream of hot process air or other suitable gas. Not typically utilized for the conventional granulation of dosage form intermediates, spray drying has gained acceptance within the industry as a robust process which can improve drug solubility and bioavailability.

Spray drying can be used to create co-precipitates of a drug/carrier which can have improved dissolution and solubility characteristics. In addition, the process can also be useful as a processing aid. For example, it is much more difficult to maintain the uniformity of a drug in suspension, as compared to the same compound in solution. One may have a need to develop an aqueous coating or drug layering process utilizing a drug which is otherwise not soluble in water. By creating a co-precipitate of the drug and a suitable water soluble carrier, often a low molecular weight polymer, the co-precipitate will remain in solution throughout the manufacturing process, improving uniformity of the spray solution and the dosage form created by the coating process. Uniformity is particularly desired where lower doses of potent compounds are intended to be coated onto beads or tablet cores.

This same process may be used to enhance the solubility and bioavailability of poorly soluble drugs. By complexing certain excipients and the active ingredient within a solvent system which is then spray dried, it is possible to enhance the drugs absorption within the body. Selection of the solvent system, the complexing agent(s) and the ratios utilized within the formulation are all formulation variables which determine the effectiveness of solubility enhancement utilizing the spray drying technique. Process parameters which also have an effect on drug solubility are the temperatures of the spray solution and process gas, the spray rate and droplet size and the rate of re-crystallization. The spray dried granulations created by these techniques can then be incorporated into capsules or tablets by conventional manufacturing processes.

### IV. METHODS OF MAKING THE EXTENDED RELEASE GASTRIC RETENTIVE DOSAGE FORMS DISCLOSED HEREIN

In one aspect, a method of making a gastric retentive extended-release dosage form as a single layer tablet comprising dry blending of the azo-bonded prodrug of 5-ASA with the binder is provided. The blended material is then granulated in the presence of water using, for example, a KitchenAid® blender. The granulated particles are then dried overnight, screened and blended with additional excipients as needed to form a mixture which is then compressed to form tablets.

Extended release polymer matrices comprising an azo-bonded prodrug of 5-ASA are made using one or a combination of one or more of the following: POLYOX® 1105 (approximate molecular weight of 900,000 Daltons), POLYOX® N-60K (approximate molecular weight of 2,000,000 Daltons), POLYOX® WSR-301 (approximate molecular weight of 4,000,000 Daltons), or POLYOX® WSR-303 (approximate molecular weight of 7,000,000 Daltons).

After granulation of the active ingredient and subsequent blending the additional excipients, batches are characterized with respect to properties such as final Loss on Drying (LOD), bulk density, tap density, and particle size.

Loss on Drying (LOD) is determined after each granulation using the Moisture Analyzer. A 1 g samples are taken and loaded into the moisture analyzer. The sample is run for 5 minutes at a temperature of 105° C.

Bulk and tap densities can be determined as follows. A graduated cylinder is filled with a certain amount of material (82-88 g), and the volume recorded to determine the material bulk density. Tap density can be determined with a help of a Tap Density Tester by exposing the material to 100 taps per test and recording the new volume.

Particle size determination is performed immediately after granulation, after sieving through 20 mesh screen to remove agglomerates. Particle diameter is determined with a sieve-type particle diameter distribution gauge using sieves with openings of 44, 53, 75, 106, 150, and 250 mesh. Fractions are weighed on Mettler balance to estimate size distribution. This provides determination of the quantitative ratio by particle diameter of composition comprising extended release particles. Sieve analysis according to standard United States Pharmacopoeia methods (e.g., USP-23 NF 18), may be done such as by using a Meinzer II Sieve Shaker.

The granulated mixture can be blended with the polymer, filler and lubricant in a V-blender. The resultant mixture can be compressed into monolithic, single-layer tablets using, for example, a Piccola Press or a Manesty® BB4 press, with the appropriate tooling.

Tablets may then be characterized with respect to disintegration and dissolution release profiles as well as tablet hardness, friability and content uniformity.

The dissolution and disintegration profiles for the tablets may be determined using a USP Dissolution apparatus I, II, or III (APP I, II, or III) or a Disintegration tester. Tests may be performed, for example, at about pH 1.2 (modified simulated gastric fluid, or mSFG), pH 4.5 (the approximate pH of the stomach after a meal) or about pH 7.5, at 37° C. Suitable aliquots of the release rate solutions are tested to determine the amount of drug released from the dosage form or pharmaceutical composition. For example, the drug can be assayed or injected into a chromatographic system to quantify the amounts of drug released during the testing intervals.

A tablet must disintegrate before it dissolves. A disintegration tester measures the time it takes a tablet to break apart in solution. The tester suspends tablets in a solution bath for visual monitoring of the disintegration rate. Both the time to disintegration and the disintegration consistency of all tablets are measured. Samples, 1 ml at each time-point, may be taken, for example, without media replacement at 0.5, 1, 2, 3, 4, 5, 6, 7 and 8 hours. The resulting cumulative disintegration profiles are based upon a theoretical percent active added to the formulation is determined.

Tablet hardness changes rapidly after compression as the tablet cools. A tablet that is too hard may not break up and dissolve into solution before it passes through the body. In the case of the presently disclosed gastric retentive dosage forms, a tablet that is too hard may not be able to imbibe fluid rapidly enough to prevent passage through the pylorus in a stomach in a fed mode. A tablet that is too soft may break apart, not handle well, and can create other defects in manufacturing. A soft tablet may not package well or may not stay together in transit.

After tablets are formed by compression, it is desired that the tablets have a strength of at least 9-25 Kiloponds (Kp)/cm², preferably at least about 12-20 (Kp)/cm². A hardness tester is used to determine the load required to diametrically break the tablets (crushing strength) into two equal halves. The fracture force may be measured using a Venkel Tablet Hardness Tester, using standard USP protocols.

Friability is a well-known measure of a tablet's resistance to surface abrasion that measures weight loss in percentage after subjecting the tablets to a standardized agitation procedure. Friability properties are especially relevant during any transport of the dosage form as any fracturing of the final dosage form will result in a subject receiving less than the prescribed medication. Friability can be determined using a Roche Friability Drum according to standard USP guidelines which specifies the number of samples, the total number of drum revolutions and the drum rpm to be used. Friability values of from 0.8 to 1.0% are regarded as constituting the upper limit of acceptability.

The prepared tablets may be tested for content uniformity to determine if they meet the pharmaceutical requirement of <6% relative standard deviation (RSD). Each tablet is placed in a solution of 1.0 N HCl and stirred at room temperature until all fragments have visibly dissolved. The solution containing the dissolved tablet is analyzed by HPLC.

In one aspect, a method of making a bilayer tablet comprising a gastric retentive extended-release layer and an immediate release layer is provided.

### V. STABILITY OF PRODRUG EXTENDED RELEASE FORMULATIONS

Stability testing is the primary tool used to assess expiration dating and storage conditions for pharmaceutical products. Many protocols have been used for stability testing, but most in the industry are now standardizing on the recommendations of the International Conference on Harmonization (ICH). These guidelines were developed as a cooperative effort between regulatory agencies and industry officials from Europe, Japan, and the United States.

Stability testing includes long-term studies, where the product is stored at room temperature and humidity conditions, as well as accelerated studies where the product is stored under conditions of high heat and humidity. Proper design, implementation, monitoring and evaluation of the studies are crucial for obtaining useful and accurate stability data. Stability studies are linked to the establishment and assurance of safety, quality and efficacy of the drug product from early phase development through the lifecycle of the drug product. Stability data for the drug substance are used to determine optimal storage and packaging conditions for bulk lots of the material. The stability studies for the drug product are designed to determine the expiration date (or shelf life). In order to assess stability, the appropriate physical, chemical, biological and microbiological testing must be performed. Usually this testing is a subset of the release testing.

Studies are designed to degrade the solid drug substance and appropriate solutions, allowing the determination of the degradation profile. The drug substance is usually challenged under a variety of accelerated environmental conditions to evaluate its intrinsic stability and degradation profile.

HPLC is the predominant tool used to analyze the drug substance and the impurities, particularly for small molecules. Frequently, the same HPLC method may be used for drug substance and drug product, although different sample preparation methods would normally be required. Often the assay and impurity testing can be performed using a single HPLC method. However, the assay and purity determinations may also be separate methods. At least in the U.S., full validation of the analytical method is not required until the end of Phase 2 clinical trials, but the establishment of specificity, linearity and limit of quantification (for impurities) is considered at the earliest stages, since verification of stability hinges on a suitable method for separating impurities from the active ingredient and at least quantifying the impurities relative to the drug substance.

Stress studies at elevated temperature (e.g., 50° C., 60° C. and 70° C.) for several weeks may be performed to assess thermal stability. Provided the degradation mechanism is the same at the different temperatures used, kinetic or statistical models can be used to determine the rate of degradation at other temperatures (e.g., 25° C.). The solid stability should also be performed in the presence and absence of water vapor to assess the dependence of stability on humidity.

Degradation studies should also be performed in solution. The solvent used for the solution testing will depend on the solubility of the drug substance and should include water, if the drug substance is water-soluble. Other solutions or solvent systems may be evaluated depending on the anticipated formulation or the synthetic process. A series of buffered solutions in the pH range 2-9 are useful in assessing the impact of solution pH on the degradation. Photostability should also be evaluated. A xenon light source can be used as a stress condition. Alternatively, one can use an accelerated version of either Options 1 or 2 as described in the ICH guideline for determination of photostability. Oxidation of the drug substance under accelerated conditions (e.g., hydrogen peroxide), may also be performed to establish oxidation products that could be formed and sensitivity to oxidative attack.

Early drug product stability studies are designed to help establish a suitable formulation for delivery of the drug substance. Compatibility studies of the drug substance with excipients should be performed to eliminate excipients that are not compatible with the drug substance.

### VI. METHODS OF TREATMENT

In another aspect, the dosage form comprising an azo-bonded prodrug of 5-ASA is administered to a subject suffering from inflammatory bowel disorders such as ulcerative colitis or other functional bowel disorders. Also envisioned are methods for treating a subject diagnosed with inflammatory bowel disease (IBD) or Crohn's disease (CD).

Generally, the frequency of administration of a particular dosage form is determined to provide the most effective results in an efficient manner without overdosing and varies according to the following criteria: (1) the characteristics of the particular drug(s), including both its pharmacological characteristics and its physical characteristics, such as solubility; (2) the characteristics of the swellable matrix, such as its permeability; and (3) the relative amounts of the drug and polymer. In most cases, the dosage form is prepared such that effective results are achieved with administration once every six hours, once every eight hours, once every twelve hours, or once every twenty-four hours. As previously discussed, due to the physical constraints placed on a tablet or capsule that is to be swallowed by a patient, most dosage forms can only support a limited amount of drug within a single dosage unit.

In one embodiment, the dosage form allows a dosing frequency of once a day (q.d.) or twice a day (b.i.d.) to provide a sustained concentration of 5-ASA at the location of the inflamed tissue of the colon, as compared to current immediate release products which require more frequent administration for effective sustained therapy.

Treatment of a subject diagnosed with a bowel disease with the gastric retained dosage form comprising an azo-bonded prodrug of 5-ASA disclosed herein can result in a decrease in the symptoms of the bowel disease. For example, once or twice-daily administration of the dosage form(s) results in a decrease in symptoms including, but not limited to, stomach pain, diarrhea and rectal bleeding caused by irritation/swelling of the colon/rectum.

To measure the efficacy of the treatment regime, subjects may be monitored for rectal bleeding. A reduction of rectal bleeding and improvement of at least one other associate symptom (stool frequency, patient functional assessment, abdominal pain, sigmoidoscopic grade, and physician's global assessment (PGA)) can be used to assess the success of treatment.

Within the context of the present disclosure, the gastric retentive dosage forms have the added advantage of improving patient compliance with administration protocols because the drugs may be administered in a once-daily or twice-daily dosing regimen, while still minimizing side effects associated with high concentrations of the prodrug or active agent (e.g., 3-ASA) which result from commercially available forms of the prodrug.

Side effects of, for example, olsalazine include headache, nausea, vomiting and/or loss of appetite. More severe side effects include diarrhea, severe stomach/abdominal pain, pale stools, unusual tiredness, persistent nausea/vomiting, change in the amount of urine, dark urine, yellowing eyes/skin, signs of infection (e.g., fever, persistent sore throat), and easy bruising/bleeding. In one embodiment of the present disclosure, a subject being administered the presently described dosage form will experience reduced side effects as compared to the subject when administered an immediate release dosage form comprising the same active agent.

For all modes of administration, the gastric retentive dosage forms described herein are preferably administered in the fed mode, i.e., with or just after consumption of a small meal (see U.S. Publication No. 2003/0104062, herein incorporated by reference).

In some aspects, the postprandial or fed mode can also be induced pharmacologically, by the administration of pharmacological agents that have an effect that is the same or similar to that of a meal. These fed-mode inducing agents may be administered separately or they may be included in the dosage form as an ingredient dispersed in the shell, in both the shell and the core, or in an outer immediate release coating. Examples of pharmacological fed-mode inducing agents are disclosed in U.S. Pat. No. 7,405,238, entitled “Pharmacological Inducement of the Fed Mode for Enhanced Drug Administration to the Stomach,” the contents of which are incorporated herein by reference.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the present disclosure or its claims.

### EXAMPLES

The following examples are intended to illustrate the dosage forms, methods of manufacture, and methods of treatment, and are not intended to limit the disclosure.

### Example 1

### Preparation of Gastric Retentive Dosage Forms Having 500 mg Olsalazine

Two formulations of extended release 500 mg olsalazine tablets comprising olsalazine and swellable polymers were manufactured using a wet granulation process. Olsalazine and polyvinylpyrrolidone (PVP) binder, approximately 6%, were mixed in a Proctor food processor and sprayed with water to generate wet granules. Granules were dried by incubating in a 50° C. oven overnight, then the dry granules were screened through a #30 mesh. Screened olsalazine-containing granules were blended with the remaining excipients in a glass jar. Tablets having a total mass of 1000 mg were hand made on a Carver Auto C Press (Fred Carver, Inc., Indiana) and compressed into tablets using a 0.4330″×0.7450″ Modified Oval die (Natoli Engineering, St. Charles, Mo.). The parameters for the operation of the Carver Auto C Press were as follows: 1500 lbs force, 0 second dwell time (the setting on the Carver Press), and 100% pump speed. Tablets had a hardness of 13-15 Kiloponds (Kp). The formulations for the tablets are set forth below in Tables 1 and 2:

TABLE 1

| | | mg/ |
|---|---|---|
| Formulation 1 | Weight % | Tablet |
| Olsalazine sodium | 50 | 500 |
| Povidone K29/32 (Plasdone, PVP) | 3 | 30 |
| SENTRY ™ POLYOX ™ WSR 1105, NF Grade | 26 | 260 |
| SENTRY ™ POLYOX ™ WSR N60K, NF Grade | 20 | 200 |
| Magnesium stearate, NF, non-bovine | 1 | 10 |
| Total | 100 | 1000 |

TABLE 2

| | | mg/ |
|---|---|---|
| Formulation 2 | Weight % | Tablet |
| Olsalazine sodium | 50 | 500 |
| Povidone K29/32 (Plasdone, PVP) | 3 | 30 |
| SENTRY ™ POLYOX ™ WSR N60K, NF Grade | 36 | 360 |
| SENTRY ™ POLYOX ™ WSR 301, NF Grade | 10 | 100 |
| Magnesium stearate, NF, non-bovine | 1 | 10 |
| Total | 100 | 1000 |

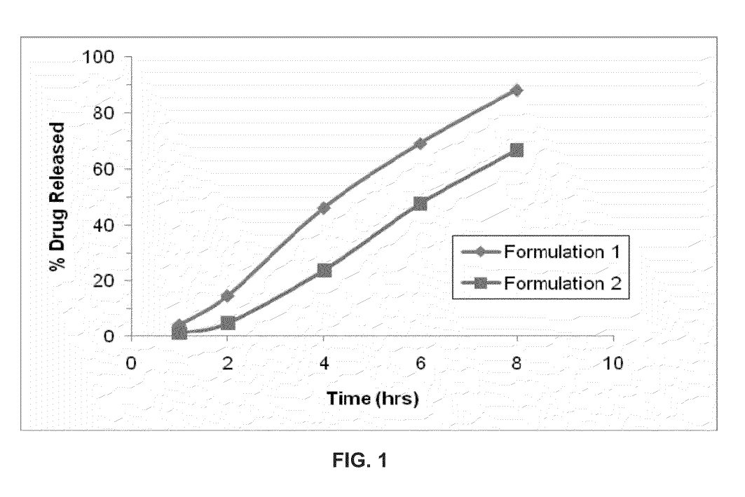

Disintegration profiles were determined in a USP Disintegration Tester in modified simulated gastric fluid, pH 1.2 at 37° C. Samples, 1 ml at each time-point, were taken without media replacement at 1, 2, 4, 6, and 8 hours. The cumulative disintegration results, based upon a theoretical percent active added to the formulation, are presented in Table 3 and the profiles are displayed in FIG. 1. The results show approximately 88% and 67% drug released after 8 hours for Formulations 1 and 2, respectively.

TABLE 3

% Drug Released

| Time (hrs) | Formulation 1 | Formulation 2 |
|---|---|---|
| 1 | 4.14 | 1.1 |
| 2 | 14.59 | 4.9 |
| 4 | 46.05 | 23.8 |
| 6 | 69.13 | 47.6 |
| 8 | 88.27 | 66.7 |

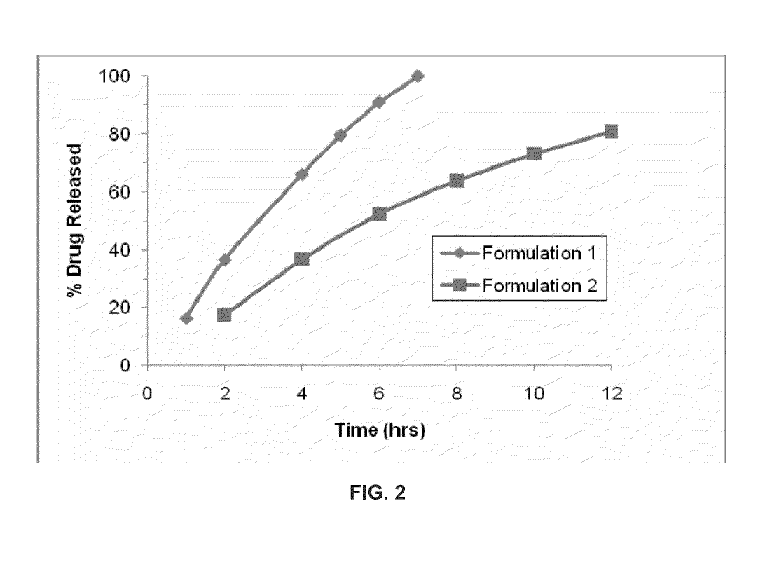

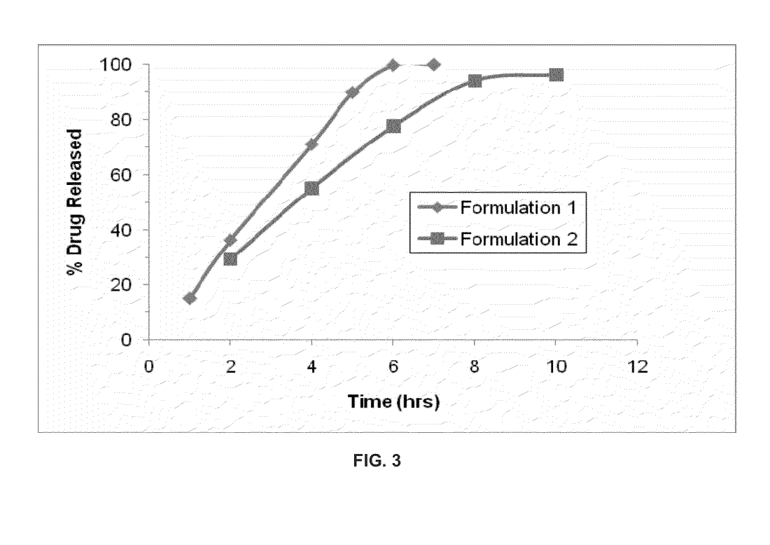

Dissolution release profiles for the tablets produced above were determined in an Apparatus III (250 ml) USP Tester in pH 4.5 acetate buffer and pH 7.5 phosphate buffer, at 37° C. Results are presented in Tables 4 and 5, and profiles are shown in FIGS. 2 and 3. At both pH 4.5 and pH 7.5, drug release from Formulation 2 is slower than from Formulation 1. For Formulation 1, at both pH 4.5 and pH 7.5, essentially all drug is released by 7 hours. For Formulation 2, drug release is sustained for approximately 10-12 hours.

TABLE 4
Formulation 1

| Time (hrs) | pH 4.5 | pH 7.5 |
|---|---|---|
| 1 | 16.08 | 15.12 |
| 2 | 36.43 | 36.21 |
| 4 | 66.08 | 71.03 |
| 5 | 79.54 | 89.88 |
| 6 | 91.03 | 99.79 |
| 7 | 100 | 100 |

TABLE 5
Formulation 2

| Time (hrs) | pH 4.5 | pH 7.5 |
|---|---|---|
| 2 | 17.5 | 29.27 |
| 4 | 36.74 | 55.1 |
| 6 | 52.46 | 77.62 |
| 8 | 63.9 | 94.22 |
| 10 | 73.16 | 96.32 |
| 12 | 80.9 | not tested |

### Example 2

### Preparation of Gastric Retentive Dosage Forms Having 750 mg Olsalazine

Two formulations of extended release 750 mg olsalazine tablets comprising olsalazine and swellable polymers were manufactured as described in Example 1. Formulations 3 and 4 were compressed into tablets using 0.4724″×0.7480″, and 0.4330″×0.7450″ Modified Oval dies (Natoli Engineering, St. Charles, Mo.), respectively. The formulations for the tablets are set forth below in Tables 6 and 7:

TABLE 6

| | | mg/ |
|---|---|---|
| Formulation 3 | Weight % | Tablet |
| Olsalazine sodium | 62.5 | 750 |
| Povidone K29/32 (Plasdone, PVP) | 3.29 | 39.5 |
| SENTRY ™ POLYOX ™ WSR N60K, NF Grade | 13.21 | 158.5 |
| SENTRY ™ POLYOX ™ WSR 301, NF Grade | 20 | 240 |
| Magnesium stearate, NF, non-bovine | 1 | 12 |
| Total | 100 | 1200 |

TABLE 7

| | | mg/ |
|---|---|---|
| Formulation 4 | Weight % | Tablet |
| Olsalazine sodium | 75 | 750 |
| Povidone K29/32 (Plasdone, PVP) | 3.95 | 39.5 |
| SENTRY ™ POLYOX ™ WSR 303, NF Grade | 20.05 | 200.5 |
| Magnesium stearate, NF, non-bovine | 1 | 10 |
| Total | 100 | 1000 |

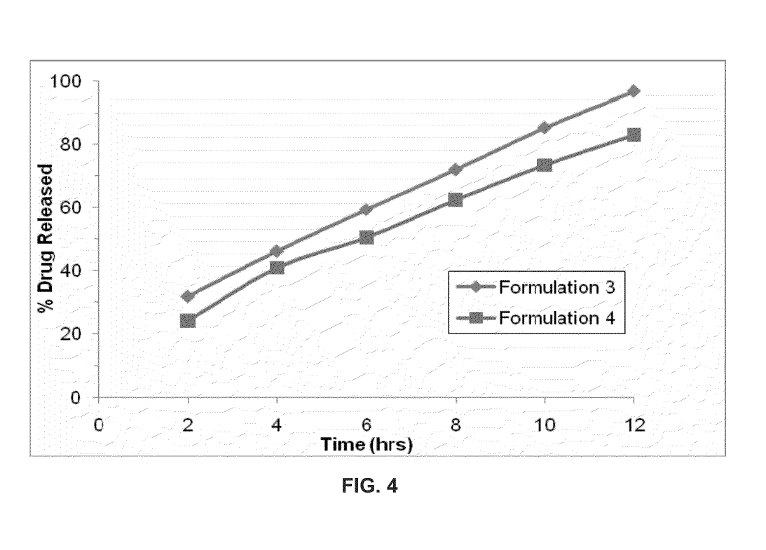

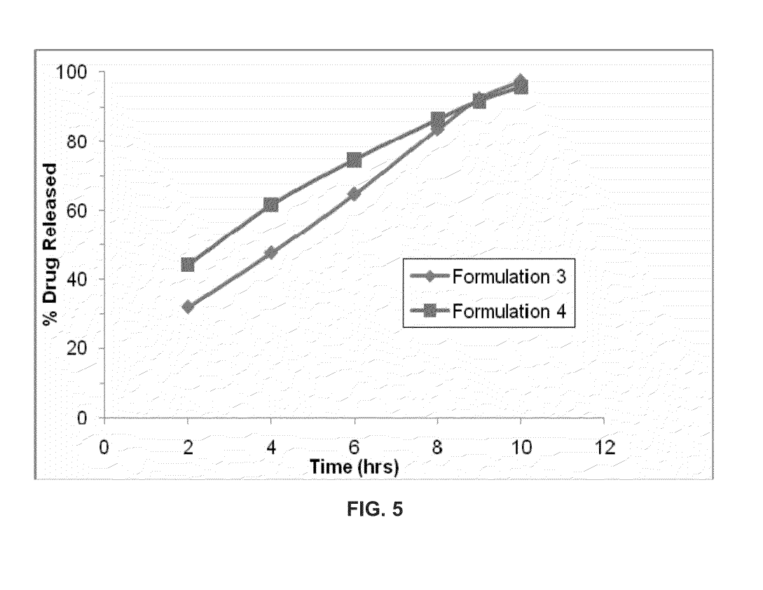

Dissolution release profiles for the tablets produced above were determined in an Apparatus III (250 ml) USP Disintegration Tester in pH 4.5 acetate buffer and pH 7.5 phosphate buffer, at 37° C. Results are presented in Tables 8 and 9, and profiles are shown in FIGS. 4 and 5. Drug release profiles are very similar for the two formulations. Release is slower at pH 4.5 than at pH 7.5 for both formulations. For Formulation 3, at both pH 4.5 and pH 7.5, essentially all drug is released by 12 hours. For Formulation 4, drug release is sustained for greater than 10 hours or more at both pH values.

TABLE 8
Formulation 3

| Time (hrs) | pH 4.5 | pH 7.5 |
|---|---|---|
| 2 | 31.8 | 32 |
| 4 | 46.2 | 47.6 |
| 6 | 59.4 | 64.7 |
| 8 | 72.1 | 83.6 |
| 9 | not tested | 92.4 |
| 10 | 85.4 | 97.5 |
| 12 | 96.8 | not tested |

TABLE 9
Formulation 4

| Time (hrs) | pH 4.5 | pH 7.5 |
|---|---|---|
| 2 | 24.1 | 44.2 |
| 4 | 40.9 | 61.6 |
| 6 | 50.5 | 74.6 |
| 8 | 62.4 | 86.4 |
| 9 | not tested | 91.7 |
| 10 | 73.5 | 95.7 |
| 12 | 83 | not tested |

### Example 3

### Swelling and Erosion Profiles of Gastric Retentive Extended-Release Olsalazine Tablets

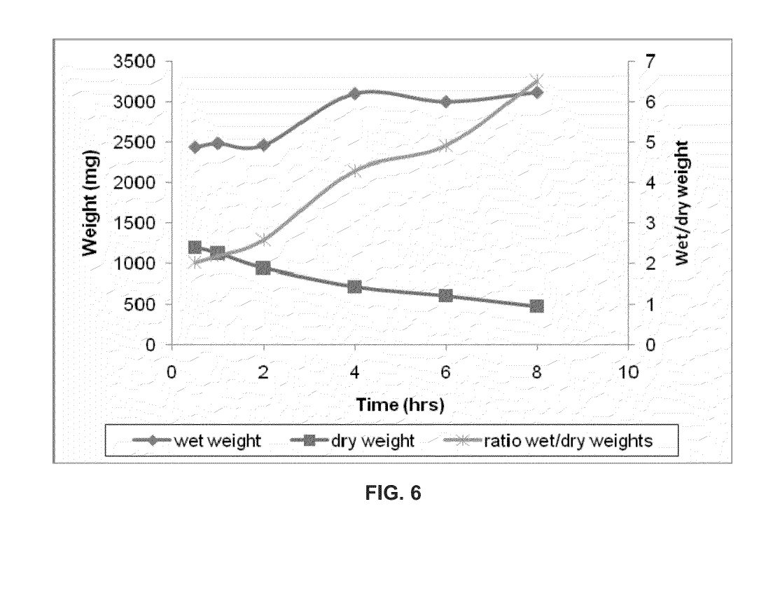

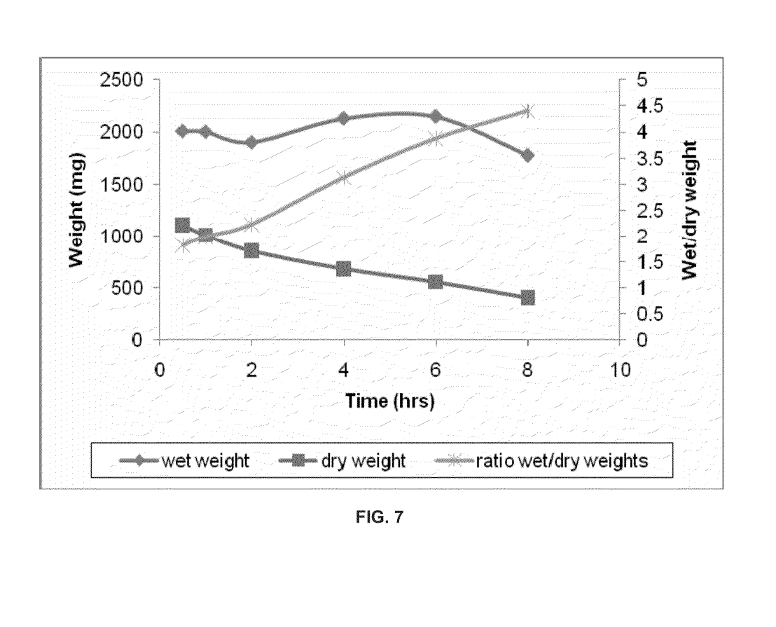

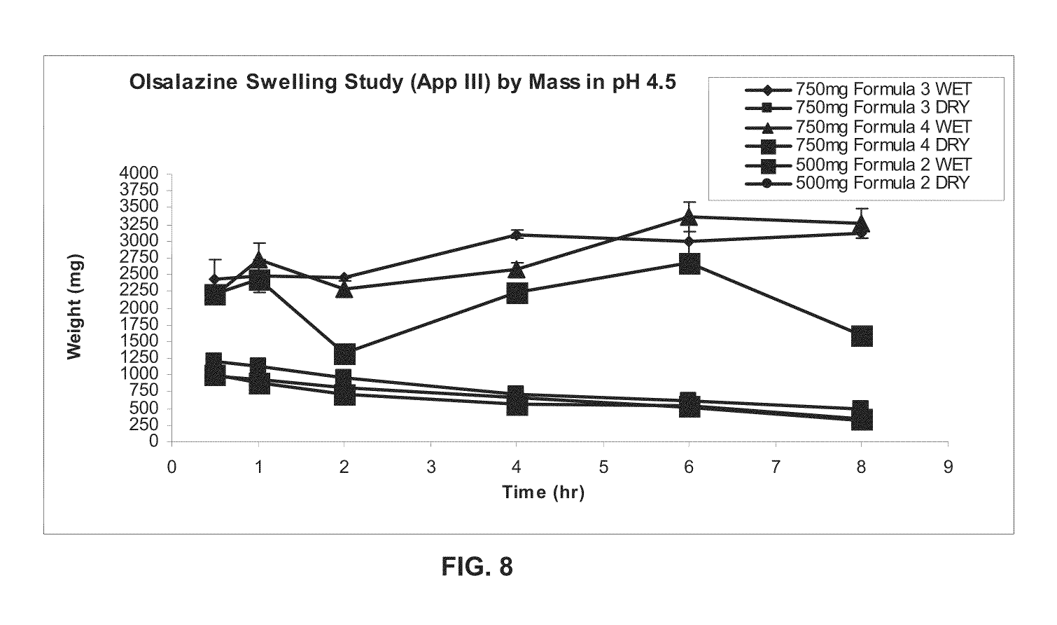

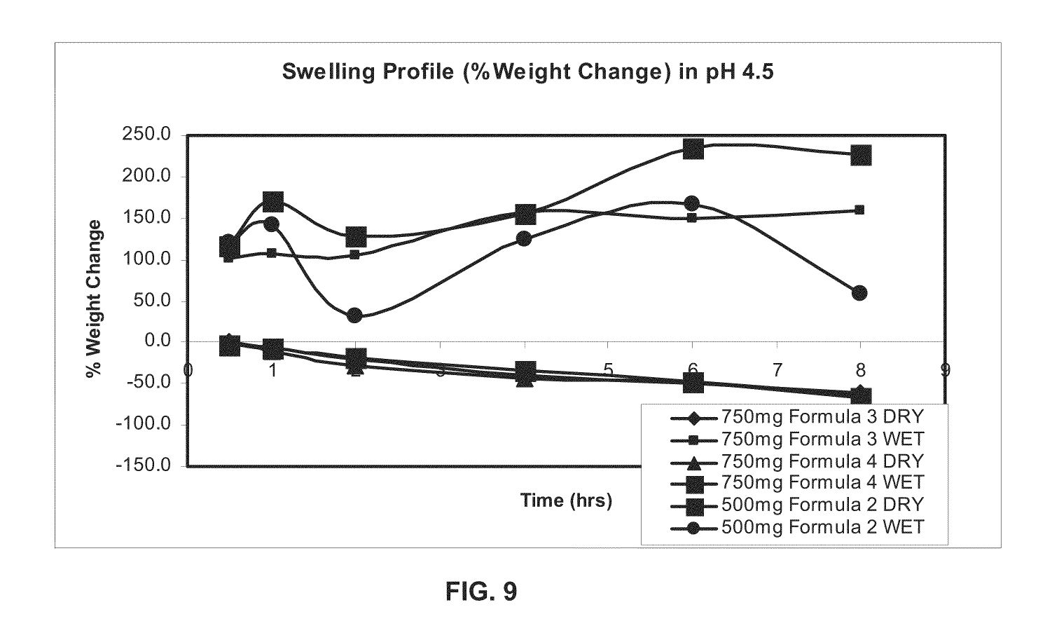

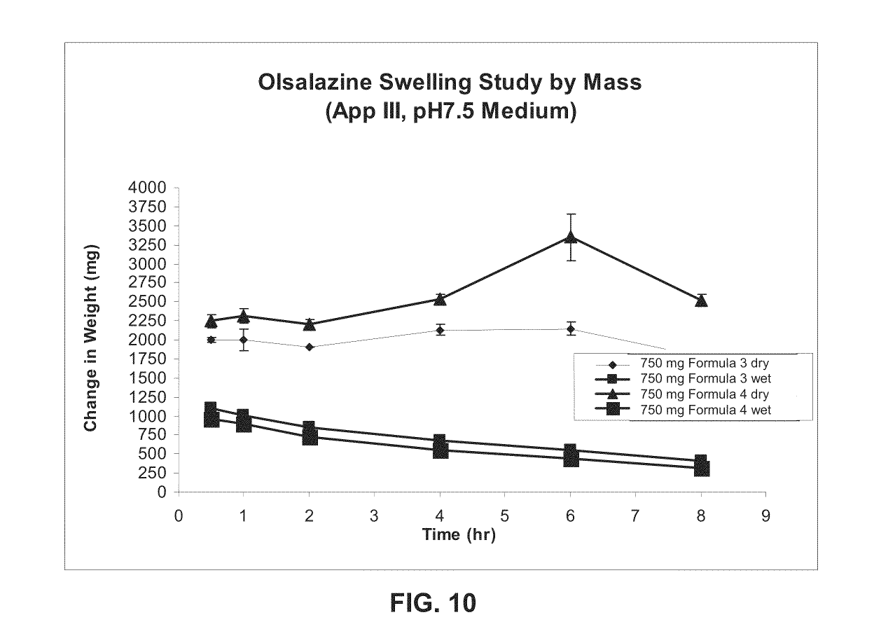

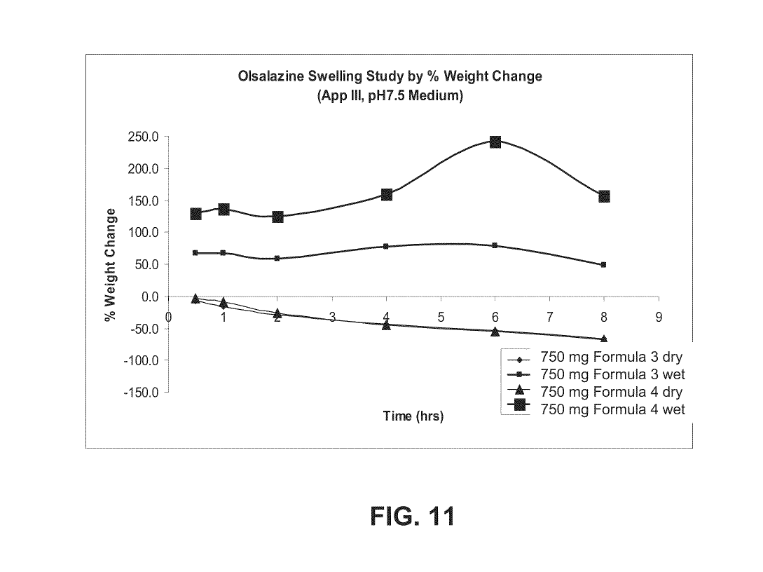

The swelling and erosion profiles of Formulation 2, Formulation 3, and Formulation 4 olsalazine tablets were assessed by monitoring weight change with time. Tablets were tested using an Apparatus III (250 ml) USP Disintegration Tester in pH 4.5 acetate buffer or in pH 7.5 phosphate buffer, at 37° C. Tablets were tested at time points between 0.5 and 8 hours. Each tablet was weighed, dried overnight at 50° C., then reweighed. The profiles are shown are shown in FIGS. 6-11. The swelling of Formulation 3 tablets is greater at pH 4.5 than at pH 7.5. The erosion rates of the tablets are similar at both pH values. As shown for Formulation 3 in FIGS. 6 and 7, the ratio of wet/dry weights changes approximately linearly with time at both pH values up to 8 hours (the last time point tested). Data for Formulation 3 are presented in Table 10.

TABLE 10

| | pH 4.5 | | | pH 7.5 | | |
|---|---|---|---|---|---|---|
| Time | wet | dry | ratio | wet | dry | ratio |
| (hrs) | (mg) | (mg) | wet/dry | (mg) | (mg) | wet/dry |
| 0.5 | 2429.33 | 1200.33 | 2.02 | 2006.33 | 1104.23 | 1.82 |
| 1 | 2477.00 | 1126.67 | 2.20 | 2000.27 | 1006.90 | 1.99 |
| 2 | 2455.00 | 947.67 | 2.59 | 1898.70 | 854.87 | 2.22 |
| 4 | 3096.33 | 720.33 | 4.30 | 2128.43 | 681.80 | 3.12 |
| 6 | 2995.67 | 610.33 | 4.91 | 2147.27 | 554.93 | 3.87 |
| 8 | 3112.67 | 478.67 | 6.50 | 1775.10 | 403.37 | 4.40 |

### Example 4

### Tablets Formulated with Micronized Olsalazine

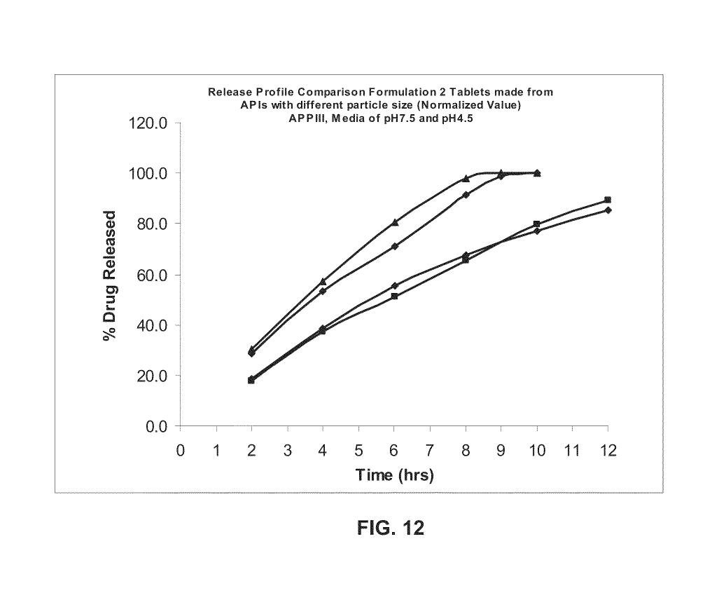

Formulation 4 tablets were also made using pharmaceutical grade olsalazine in a micronized form in order to test the effects of drug particle size on tablet manufacturing and the ensuing release profiles. In vitro release profiles were evaluated for Formulation 4 tablets made as described in Example 1 and for tablets made using the same formula and process except with micronized olsalazine. Dissolution profiles were determined in pH 7.5 phosphate buffer because olsalazine dissolves easily in this media, and in pH 4.5 acetate buffer to simulate the conditions of the gastrointestinal tract. Olsalazine did not completely dissolve in the pH 4.5 media, so the resultant dissolution sample was adjusted for higher pH of 7.5 to achieve complete dissolution of the drug. As shown in FIG. 12, the Formulation 4 tablets made from granules of standard and micronized particle size show similar in vitro release profiles in the pH of 7.5 phosphate buffer media and pH 4.5 acetate buffer media, suggesting that the olsalazine API does not significantly impact the in vitro drug release profile.

### Example 5

### Erosion Studies with Gastric Retentive Extended-Release Olsalazine Tablets in Beagle Dogs

This study was conducted in 4 or 5 healthy female beagle dogs weighing between 12-16 kg to determine the erosion time of 4 formulations of gastric retentive extended-release olsalazine tablets. Following an overnight fast of at least 14 hours, the dogs were fed 100 g of canned dog food (Pedigree® Traditional ground Dinner with Chunky Chicken). Within 15 minutes of the dog consuming the meal they were administered one of the olsalazine tablet formulations (Formulations 1-4). Each dog received each formulation except Dog #3 who only received Formulation 1 and Formulation 2. There were at least 2 days between administrations of Formulations 1 and 2, and then a period of about 6 month elapsed before Formulations 3 and 4 were administered. Erosion of the gastric retentive extended-release tablets was assessed using fluoroscopy. Each tablet contained two radio-opaque strings in the shape of an “X”. Separation of the strings was considered to signify complete erosion of the tablets. Images were obtained every 30 minutes until the strings separated. The recorded erosion time was the midpoint between the last time the tablet was intact and when the strings were separated.

The erosion times for Formulation 2 were longer than for Formulation 1, and the erosion times of Formulation 2 (500 mg dose) were similar to those of Formulation 3 (750 mg dose). From the dog erosion study a prediction of delivery (erosion) time was calculated based on a correlation developed between dog and human from previous erosional tablet studies.

The dog erosion results and predicted human erosion times (delivery times) are listed in Tables 11 and 12.

TABLE 11

| | Formulation 1 | | Formulation 2 | |
|---|---|---|---|---|
| | | Predicted | | Predicted |
| | Dog erosion | Human erosion | Dog erosion | Human erosion |
| Dog # | time (hrs) | time (hrs) | time (hrs) | time (hrs) |
| 1 | 3.75 | 6.93 | 5.75 | 9.93 |
| 2 | 4.75 | 8.43 | 5.25 | 9.18 |
| 3 | 3.25 | 6.18 | 4.75 | 8.43 |
| 4 | 4.25 | 7.68 | 7.25 | 12.18 |
| 5 | 3.75 | 6.93 | 6.25 | 10.68 |
| Mean ± | 3.95 ± 0.57 | 7.23 ± 0.86 | 5.85 ± 0.96 | 10.08 ± 1.44 |
| SD | | | | |

TABLE 12

| | Formulation 3 | | Formulation 4 | |
|---|---|---|---|---|
| | | Predicted | | Predicted |
| | Dog erosion | Human erosion | Dog erosion | Human erosion |
| Dog # | time (hrs) | time (hrs) | time (hrs) | time (hrs) |
| 1 | 4.75 | 8.43 | 4.75 | 4.75 |
| 2 | 4.75 | 8.43 | 4.25 | 4.25 |
| 3 | ND | ND | ND | ND |
| 4 | 5.25 | 9.18 | 2.75 | 2.75 |
| 5 | 5.25 | 9.18 | 3.75 | 3.75 |
| Mean ± | 5.00 ± 0.29 | 8.80 ± 0.43 | 3.88 ± 0.85 | 7.11 ± 1.28 |
| SD | | | | |

ND = not done as dog had been removed from study group

Certain modifications and improvements will occur to those skilled in the art upon a reading of the foregoing description. It should be understood that all such modifications and improvements have been deleted herein for the sake of conciseness and readability but are properly within the scope of the following claims.

1. A gastric retentive dosage form comprising
a gastric retentive (GR) layer comprising an azo-bonded prodrug of 5-ASA dispersed in a polymeric matrix wherein the polymeric matrix comprises one or more polymers that upon imbibition of fluid swells to a size sufficient to promote gastric retention.

2. The dosage form of claim 1, wherein the azo-bonded prodrug of 5-ASA is olsalazine, sulfasalazine or balsalazide, or a pharmaceutically acceptable salt thereof.

3. The dosage form of claim 1, wherein the GR layer comprises about 200 mg to 1000 mg of the azo-bonded prodrug of 5-ASA.

4. The dosage form of claim 1, wherein the prodrug is released from the tablet over a time period of about 5 hours to 13 hours.

5. The dosage form of claim 1, wherein not less than 60% of the prodrug is released from the dosage form within about 6 hours after contact of the dosage form with a fluid.

6. The dosage form of claim 1, wherein about 5% to 20% of the prodrug is released from the dosage form without about 2 hours after contact of the dosage form with a fluid.

7. The dosage form of claim 1, wherein the dosage form swells upon imbibition of fluid to a size which is at least about 20% larger than the size of the tablet prior to imbibition of fluid.

8. The dosage form of claim 1, wherein the polymeric matrix comprises at least one hydrophilic polymer which has an approximate molecular weight of 900,000 Daltons (Da) to 10,000,000 Da.

9. A method of treatment comprising administering to a subject in need thereof a gastric retentive dosage form which comprises an azo-bonded prodrug of 5-ASA, wherein the prodrug is dispersed in a polymeric matrix wherein the polymeric matrix comprises one or more polymers that upon imbibition of fluid swells to a size sufficient to promote gastric retention.

10. The method of claim 9, wherein the subject has been diagnosed with an inflammatory bowel disorder.

11. The method of claim 9, wherein the subject has been diagnosed with ulcerative colitis. Crohn's disease or inflammatory bowel disease.

12. The method of claim 9, wherein the subject experiences a reduction in rectal bleeding after administering for a period of at least about 1, 2 or 3 days.

13. The method of claim 9, wherein the prodrug is olsalazine, sulfasalazine or balsalazide, or a pharmaceutically acceptable salt thereof.

14. The method of claim 9, wherein the administering comprises oral administration of the dosage form once- or twice-daily.

15. The method of claim 9, wherein the administering comprises oral administration of a total of about 250 mg to 10 g of the prodrug within a 24-hour period.

16. The method of claim 9, wherein the administering comprising administering the dosage form to the subject in a fed state.
